(19) United States
(12) Patent Application Publication
(10) Pub. No.: US 2025/0186193 A1
(43) Pub. Date: Jun. 12, 2025

(54) PROSTHETIC VALVE DOCKING DEVICES

(71) Applicant: EDWARDS LIFESCIENCES CORPORATION, Irvine, CA (US)

(72) Inventor: Evan T. Schwartz, Huntington Beach, CA (US)

(21) Appl. No.: 19/061,054

(22) Filed: Feb. 24, 2025

Related U.S. Application Data
(63) Continuation of application No. PCT/US2023/031250, filed on Aug. 28, 2023.
(60) Provisional application No. 63/374,343, filed on Sep. 1, 2022.

(51) Int. Cl. A61F 2/24

(52) U.S. Cl. CPC A61F 2/2409; A61F 2220/0008; A61F 2220/0075; A61F 2230/0091; A61F 2250/001; A61F 2250/0063; A61F 2250/0098

(57) ABSTRACT

Docking devices for securing a prosthetic valve at a native valve and delivery apparatus for delivery thereof. The docking device includes a coil member that can be moved into a coiled configuration, such that the coil member includes a plurality of helical turns when deployed at the native valve. The guard member includes a plurality of fibers that can extend radially outward from an exterior surface of the coil. The guard member can be secured to the coil via one or more sutures. The guard member can be compressed within a dock sleeve during delivery of the docking device to the native valve. The dock sleeve can be withdrawn or axially moved relative to the coil member to allow the guard member to transition from the compressed state to the radially extended state. The guard member can function anchor a prosthetic valve and limit or prevent paravalvular leakage when implanted in a native heart valve.

### CROSS-REFERENCE TO RELATED APPLICATIONS

This application is a continuation of PCT Patent Application No. PCT/US2023/031250, filed on Aug. 28, 2023, which application claims the benefit of U.S. Provisional Patent Application No. 63/374,343, filed on Sep. 1, 2022, each of which application being incorporated by reference herein in its entirety.

### FIELD

The present disclosure concerns examples of a docking device configured to secure a prosthetic valve at a native heart valve, as well as apparatus and methods for delivery of such docking devices.

### BACKGROUND

Prosthetic valves can be used to treat cardiac valvular disorders. Native heart valves (e.g., the aortic, pulmonary, tricuspid and mitral valves) function to prevent backward flow or regurgitation, while allowing forward flow. These heart valves can be rendered less effective by congenital, inflammatory, infectious conditions, etc. Such conditions can eventually lead to serious cardiovascular compromise or death. For many years, the doctors attempted to treat such disorders with surgical repair or replacement of the valve during open heart surgery.

A transcatheter technique for introducing and implanting a prosthetic heart valve using a catheter in a manner that is less invasive than open heart surgery can reduce complications associated with open heart surgery. In this technique, a prosthetic valve can be mounted in a compressed state on the end portion of a catheter and advanced through a blood vessel of the patient until the valve reaches the implantation site. The valve at the catheter tip can then be expanded to its functional size at the site of the defective native valve, such as by inflating a balloon on which the valve is mounted or, for example, the valve can have a resilient, self-expanding stent or frame that expands the valve to its functional size when it is advanced from a delivery sheath at the distal end of the catheter. Optionally, the valve can have a balloon-expandable, self-expanding, mechanically expandable frame, and/or a frame expandable in multiple or a combination of ways.

In some instances, a transcatheter heart valve (THV) may be appropriately sized to be placed inside a particular native valve (e.g., a native aortic valve). As such, the THV may not be suitable for implantation at another native valve (e.g., a native mitral valve) and/or in a patient with a larger native valve. Additionally or alternatively, the native tissue at the implantation site may not provide sufficient structure for the THV to be secured in place relative to the native tissue. Accordingly, improvements to THVs and the associated transcatheter delivery apparatus are desirable.

### SUMMARY

Described herein are examples of docking devices and delivery apparatus that can be used in combination with prosthetic heart valves. In examples, the docking device can include a coil member configured to be transitioned from a delivery configuration to a coiled configuration wherein the coil member forms a plurality of helical turns. Further, in examples, the docking device includes a guard member that extends circumferentially over at least a portion of one or more of the helical turns when the coil member is in the coiled configuration, and the guard member is configuration to be transitioned between a delivery (compressed) configuration and a deployed (expanded or extended) configuration. When the docking device is implanted in the coiled configuration and the guard member is transitioned to the expanded state, the guard member can help prevent, limit, or reduce paravalvular leakage (PVL) or regurgitation and/or promote tissue ingrowth between the native tissue and the docking device.

A prosthetic heart valve can comprise a frame and a valvular member coupled to the frame. In addition to these components, a prosthetic heart valve can further comprise one or more of the valve components disclosed herein.

A docking device for use with a prosthetic heart valve can comprise a coil member that can be deployed into a coiled configuration. In addition to the coil member, a docking device can further comprise one or more of the docking device components disclosed herein.

In some examples, the coiled configuration comprises a plurality of helical turns.

In some examples, the docking device is configured to transition from a delivery configuration to the coiled configuration.

In some examples, the docking device can further include a guard member.

In some examples, the guard member can extend circumferentially over at least a portion of each of one or more of the helical turns relative to a central longitudinal axis of the coil member in the coiled configuration.

In some examples, the guard member can include a plurality of fibers configured to be transitioned from a compressed state and a radially extended state.

In some examples, the guard member can extend over an exterior surface of the at least portion of one or more the helical turns of the coil members.

In some examples, the guard member can extend over an exterior surface of one of the helical turns of the coil members.

In some examples, the guard member can be attached to the coil member via sutures.

In some examples, the guard member can be attached to the coil member via a plurality of sutures that are knotted around the guard member and the coil member at specified increments.

In some examples, the guard member can be attached to the coil member via a wrap suture that wraps around the guard member and the coil member at specified increments.

In some examples, when the guard member is in the radially extended state, the plurality of fibers can extend radially outwardly from an exterior surface of the coil member.

In some examples, when the guard member is in the compressed state, the plurality of fibers can be compressed against the exterior surface of the coil member by a dock sleeve of a delivery apparatus.

In some examples, the coil member in the coiled configuration can be transitioned from a first radially expanded state to a second radially expanded state via expansion of the prosthetic valve within a central space of the helical turns of the coil member.

In some examples, the coil member can have a first diameter in the first radially expanded state and has a second greater diameter in the second radially expanded state.

In some examples, the guard member can extend around the coil member in the coiled configuration to a lesser degree when the coil member is in the second radially expanded state relative to the coil member in the second radially expanded state.

In some examples, in the first radially expanded state, the guard member can extend circumferentially more than 360 degrees around the coil member in the coiled configuration.

In some examples, in the second radially expanded state, the guard member can extends circumferentially less than 361 degrees around the coil member in the coiled configuration.

In some examples, the docking device can include one or more seating markers disposed at a proximal region of a proximal turn of the coil member.

In some examples, one or more seating markers can include a proximal seating marker and a distal seating marker, and a proximal end of the guard member can be disposed between the proximal seating marker and the distal seating marker.

In some examples, the guard member can be discontinuous and comprises two sections disposed on opposing sides of the coil member in the coiled configuration.

In some examples, the guard member comprises at least two intertwined axial core members and the plurality of fibers are retained between the at least two intertwined axial core members.

In some examples, the guard member comprises a base layer, and the fibers are integral with and extend from the base layer or are attached to the base layer.

A delivery apparatus for a prosthetic implant can comprise a handle and one or more shafts coupled to the handle.

A delivery apparatus for a docking device can comprise a handle and one or more shafts coupled to the handle.

In some examples, the delivery apparatus can comprise include a dock sleeve for compressing fibers of the guard member against the exterior surface of the coil member.

In some examples, a dock sleeve can be configured to circumferentially and axially extend over the coil member at a region corresponding to a location of the guard member and retain the plurality of fibers into the compressed state, and the dock sleeve can be moved axially relative to the coil member to release the plurality of fibers and allow the fibers to transition from the compressed state to the radially extended state.

In some examples, the dock sleeve can be retracted relative to the coil member to transition the guard member from a compressed state to a radially extended state.

An assembly can include a prosthetic valve having one or more of the valve components described herein and a docking device having one or more of the docking device components described herein.

In some examples, the assembly additionally includes a delivery apparatus having one or more of the delivery apparatus components described herein.

A method for implanting a prosthetic implant can include deploying, into a coiled configuration, a docking device having a guard member at a native valve and transitioning the plurality of fibers of the guard member from a compressed state to a radially extended state.

In some examples, the method can further include deploying the prosthetic valve within a central space of the coil of the docking device.

In some examples, deploying the prosthetic valve comprises radially expanding the prosthetic valve, which can result in transitioning of the coil of the docking device from a first radially expanded state to a second radially expanded state, the coil having a first diameter in the first radially expanded state and a second diameter in the second radially expanded state, the second diameter greater than the first diameter.

In some examples, relative to a longitudinal axis of the coil, the guard member can extend more than 360 degrees circumferentially around the coil when the coil is in the first radially expanded state and can extend approximately 360 degrees circumferentially around the coil when the coil is in the second radially expanded state.

The above method(s) can be performed on a living animal or on a simulation, such as on a cadaver, cadaver heart, anthropomorphic ghost, simulator (e.g., with body parts, heart, tissue, etc. being simulated).

In some examples, a docking device, an assembly, and/or a method of use thereof can comprises one or more of the components recited in Examples 1-63 below.

In one representative example, a docking device configured for securing a prosthetic valve includes a coil member configured to be transitioned from a delivery configuration to a coiled configuration, wherein the coil member, when in the coiled configuration, comprises a plurality of helical turns. In some examples, the docking device further includes a guard member extending, relative to a central longitudinal axis of the coil member in the coiled configuration, circumferentially over at least a portion of one or more of the helical turns, wherein the guard member is configured to be transitioned between a compressed state and an expanded state. In some examples, the guard member comprises a plurality of fibers that extend radially from a surface of the coil member when the guard member is in the expanded state. In some examples, the further comprises at least two intertwined axial core members, and the plurality of fibers are retained between the at least two axial core members. In some examples, the guard member further comprises a base layer, and the plurality of fibers attached to or extending from the base layer.

In another representative example, an assembly includes a prosthetic valve comprising an annular frame configured to be transitioned from a radially compressed configuration and a radially expanded configuration and a valvular member disposed within the annular frame. In some examples, the assembly further includes a docking device configured for anchoring the prosthetic valve, the docking device including a coil member configured to be transitioned from a delivery configuration to a coiled configuration and the coil member including a plurality of helical turns when the coil member is in the coiled configuration. In some examples, the docking device further includes a guard member extending, relative to a central longitudinal axis of the coil member in the coiled configuration, circumferentially over at least a portion of one or more of the helical turns, the guard member including a plurality of fibers configured to be transitioned between a compressed state and a radially extended state. In some examples, the plurality of core members are retained between at least two axial core members. In some examples, the plurality of core members are attached to or extend from a base layer.

In another representative example, a delivery assembly includes a docking device configured for anchoring a prosthetic valve, where the docking device includes a coil member configured to be transitioned from a delivery configuration to a coiled configuration, the coil member, when in the coiled configuration, comprising a plurality of helical turns. In some examples, the docking device further comprises a guard member extending circumferentially relative to a central longitudinal axis of the coil member in the coiled configuration over at least a portion of each of one or more of the helical turns. In some examples, the guard member comprises a plurality of fibers retained between at least two intertwined axial core members or attached to or extending from a base layer, and the plurality of fibers are configured to be transitioned between a compressed state and radially extended state. In some examples, the exemplary delivery assembly further includes a docking device delivery apparatus configured for transcatheter delivery of the docking device to a native heart valve, the docking device delivery apparatus comprising a delivery shaft comprising a lumen configured to receive the coil member therethrough when the coil member is in the delivery configuration, and a dock sleeve configured to circumferentially and axially extend over at least a portion of the coil member corresponding to a location of the guard member and compress the plurality of fibers into the compressed state. In some examples, the dock sleeve is further configured to move axially relative to the coil member to release the plurality of fibers and/or allow the fibers to transition from the compressed state to the radially extended state.

In yet another representative example, a method for implanting a prosthetic implant includes deploying a docking device at a native valve, wherein the docking device deployed at the native valve comprises a coil and a guard member extending circumferentially around at least a portion of the coil on an exterior surface thereof, the guard member comprising a plurality of fibers; and transitioning the plurality of fibers of the guard member from a compressed state to a radially extended state.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

The foregoing and other objects, features, and advantages of the invention will become more apparent from the following detailed description, which proceeds with reference to the accompanying figures.

### BRIEF DESCRIPTION OF THE DRAWINGS

FIG. 1 schematically illustrates a first stage in an exemplary mitral valve replacement procedure where a guide catheter and a guidewire are inserted into a blood vessel of a patient and navigated through the blood vessel and into a heart of the patient, towards a native mitral valve of the heart.

FIG. 2A schematically illustrates a second stage in the exemplary mitral valve replacement procedure where a docking device delivery apparatus extending through the guide catheter is implanting a docking device for a prosthetic heart valve at the native mitral valve.

FIG. 2B schematically illustrates a third stage in the exemplary mitral valve replacement procedure where the docking device of FIG. 2A is fully implanted at the native mitral valve of the patient and the docking device delivery apparatus has been removed from the patient.

FIG. 3A schematically illustrates a fourth stage in the exemplary mitral valve replacement procedure where a prosthetic heart valve delivery apparatus extending through the guide catheter is implanting a prosthetic heart valve in the implanted docking device at the native mitral valve.

FIG. 3B schematically illustrates a fifth stage in the exemplary mitral valve replacement procedure where the prosthetic heart valve is fully implanted within the docking device at the native mitral valve and the prosthetic heart valve delivery apparatus has been removed from the patient.

FIG. 4 schematically illustrates a sixth stage in the exemplary mitral valve replacement procedure where the guide catheter and the guidewire have been removed from the patient.

FIG. 5A is a perspective view of an exemplary delivery apparatus and a prosthetic heart valve that can be used in the exemplary mitral valve replacement procedure illustrated in FIG. 1-4, according to one example.

FIG. 5B is a side view of an exemplary delivery apparatus and a docking device that can be used in the exemplary mitral valve replacement procedure illustrated in FIG. 1-4, according to one example.

FIG. 6A is a side perspective view of a docking device in a helical configuration, according to one example.

FIG. 6B is a top view of the docking device depicted in FIG. 6A.

FIG. 7A is a cross-sectional view of the docking device taken along line A-A depicted in FIG. 6B, according to one example.

FIG. 7B is a cross-sectional view of the docking device taken along the same line as in FIG. 7A, except in FIG. 7B the docking device is in a delivery configuration and has a dock sleeve disposed therearound.

FIG. 7C is a schematic illustration of an exemplary configuration for a guard member for use with the docking devices of FIGS. 6A and 6B.

FIGS. 7D and 7E are side views of exemplary guard members for use with the docking devices of FIGS. 6A and 6B.

FIG. 8A is a cross-sectional view of the docking device taken along line A-A depicted in FIG. 6B, according to one example.

FIG. 8B is a cross-sectional view of the docking device taken along the same line as in FIG. 8A, except in FIG. 8B the docking device is in a delivery configuration and has a dock sleeve disposed therearound.

FIGS. 8C and 8D are side views of exemplary guard members for use with the docking devices of FIGS. 6A and 6B.

FIGS. 9A and 9B are schematic illustrations of exemplary suture patterns for attachment of one of the guard members of FIGS. 7C-7E and 8C-8D to a docking device.

FIG. 10A is a perspective view a prosthetic valve, according to one example.

FIG. 10B is a perspective view of the prosthetic valve of FIG. 10A with an outer cover, according to one example.

FIGS. 11A and 11B are respectively top and side perspective views a docking device in a helical configuration and with the prosthetic valve of FIGS. 10A and 10B radially expanded within a central space of the docking device.

FIG. 12A is an atrial side view of a docking device implanted in the mitral valve, according to one example.

FIG. 12B is a ventricle side view of the implanted docking device shown in FIG. 12A.

FIG. 13A is an atrial side view of a docking device implanted in the mitral valve, according to another example.

FIG. 13B is a ventricle side view of the implanted docking device shown in FIG. 13A.

### DETAILED DESCRIPTION

### General Considerations

It should be understood that the disclosed examples can be adapted to deliver and implant prosthetic devices in any of the native annuluses of the heart (e.g., the mitral and tricuspid annuluses), and can be used with any of various delivery approaches (e.g., retrograde, antegrade, transseptal, transventricular, transatrial, etc.).

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the terms “coupled” and “connected” generally mean electrically, electromagnetically, and/or physically (e.g., mechanically or chemically) coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

As used herein, the term “approximately” and “about” means the listed value and any value that is within 10% of the listed value. For example, “about 1 mm” means any value between about 0.9 mm and about 1.1 mm, inclusive.

Directions and other relative references (e.g., inner, outer, upper, lower, etc.) may be used to facilitate discussion of the drawings and principles herein, but are not intended to be limiting. For example, certain terms may be used such as “inside,” “outside,”, “top,” “down,” “interior,” “exterior,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same. As used herein, “and/or” means “and” or “or,” as well as “and” and “or.”

As used herein, “e.g.” means “for example,” and “i.e.” means “that is.”

### Introduction to the Disclosed Technology

Disclosed herein are various systems, apparatus, methods, etc., including anchoring or docking devices, which can be used in conjunction with expandable prosthetic valves at a native valve annulus (e.g., a native mitral and/or tricuspid valve annulus), in order to more securely implant and hold the prosthetic valve at the implant site. Anchoring/docking devices according to examples of the disclosure can, for example, provide a stable anchoring site, landing zone, or implantation zone at the implant site in which prosthetic valves can be expanded or otherwise implanted. Many of the disclosed docking devices comprise a coil member that can be moved into a circular or coiled or cylindrically-shaped configuration, which can (for example) allow a prosthetic heart valve comprising an annular or cylindrically-shaped valve frame or stent to be expanded or otherwise implanted into native locations with naturally circular cross-sectional profiles and/or in native locations with naturally with non-circular cross-sections.

In some examples, in addition to providing an anchoring site for the prosthetic valve, the anchoring/docking devices can be sized and shaped to cinch or draw the native valve (e.g., mitral, tricuspid, etc.) anatomy radially inwards. In this manner, one of the main causes of valve regurgitation (e.g., functional mitral regurgitation), specifically enlargement of the heart (e.g., enlargement of the left ventricle, etc.) and/or valve annulus, and consequent stretching out of the native valve (e.g., mitral, etc.) annulus, can be at least partially offset or counteracted. Some examples of the anchoring or docking devices further include features which, for example, are shaped and/or modified to better hold a position or shape of the docking device during and/or after expansion of a prosthetic valve therein. By providing such anchoring or docking devices, replacement valves can be more securely implanted and held at various valve annuluses, including, for example, at the mitral valve annulus which does not have a naturally circular cross-section.

In some instances, a docking device can comprise a paravalvular leakage (PVL) guard (also referred to herein as “a guard member”). The PVL guard can, for example, help reduce regurgitation and/or promote tissue ingrowth between the native tissue and the docking device.

The PVL guard can, in some examples, be transitioned between a compressed delivery configuration or state and an expanded, released, freed, or extended deployment configuration or state. In some examples, the PVL guard can include fibers or pile yarns that extend radially outward from a surface of the docking device when the PVL guard is in the released state. In some examples, plurality of fibers are retained between two intertwined axial core members. In some examples, the plurality of fibers are attached to or extend from a surface of a base layer (such as, for example, a woven material base layer).

When the PVL guard is in the delivery configuration, the fibers or pile yarns can be disposed inside of a dock sleeve and compressed against exterior surface of the coil member. When the PVL guard is in the deployed configuration, the dock sleeve is removed which can enable the fibers to be released or freed, and can further enable the plurality of fibers to extend radially outward from at least a portion of the surface of the coil member. In some examples, when implanted, the guard member can extend radially outward from the exterior surface of the coil member in the coiled configuration over a portion of a turn in the coil or over one or more turns in the coil.

In some examples, the PVL guard can be a continuous cover extending over an exterior surface of one or more turns in a coil of the docking device. For example, the PVL guard can extend between 180-540 degrees circumferentially relative to a longitudinal axis of the coil member in the coiled configuration. In some examples, the PVL guard can be discontinuous, and cover discrete portions of an exterior surface of one or more turns in a coil of the docking device. For example, the PVL guard can include two sections disposed on opposing sides of one of the turns of the coil member in the coiled configuration. In some examples, the PVL guard can surround or encircle the coil member, thereby covering interior and exterior surfaces of one or more turns of the coil of the docking device.

Exemplary methods of attaching a PVL guard to a docking device and example methods of implanting a docking device including a PVL guard are also disclosed herein.

### Examples of the Disclosed Technology

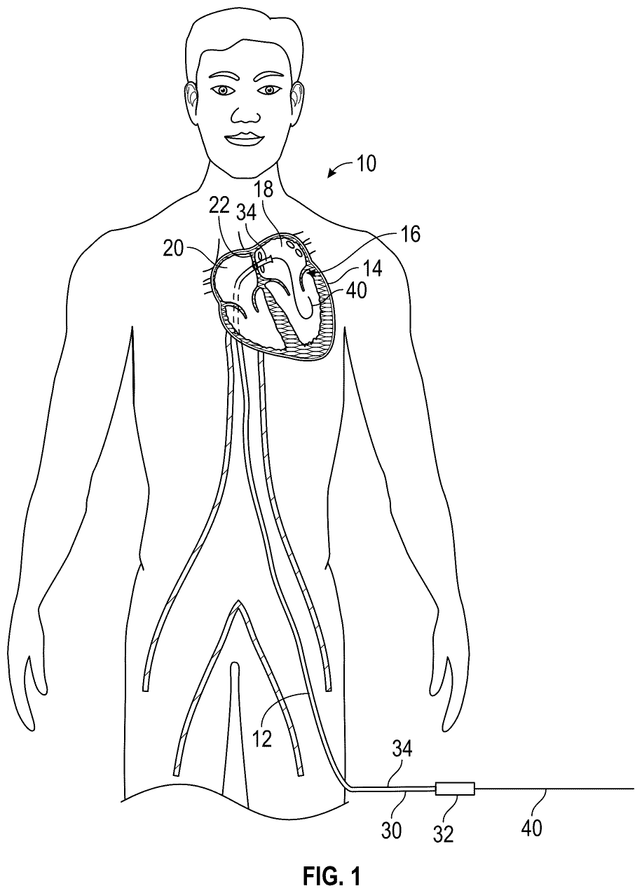

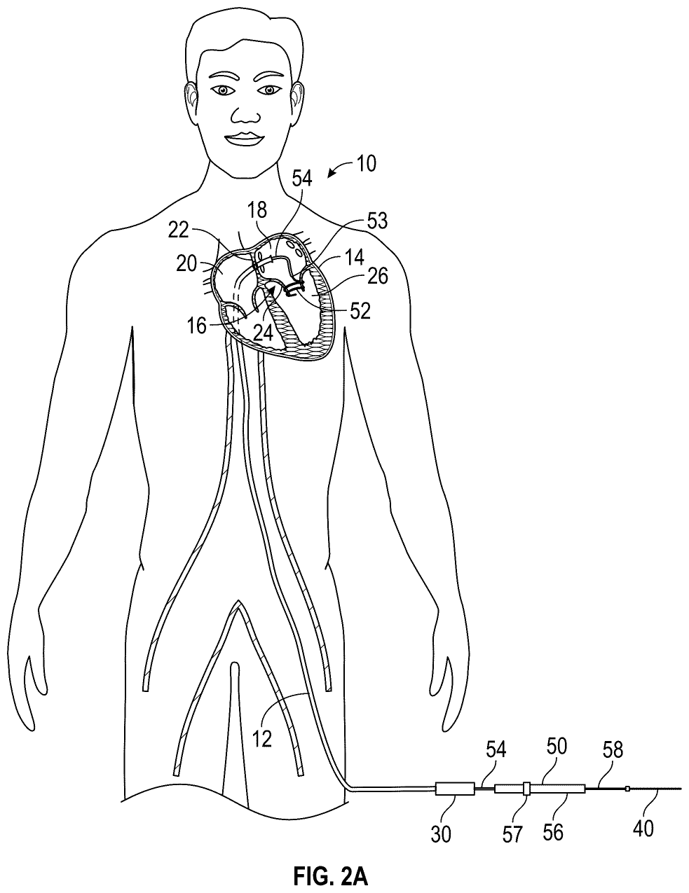

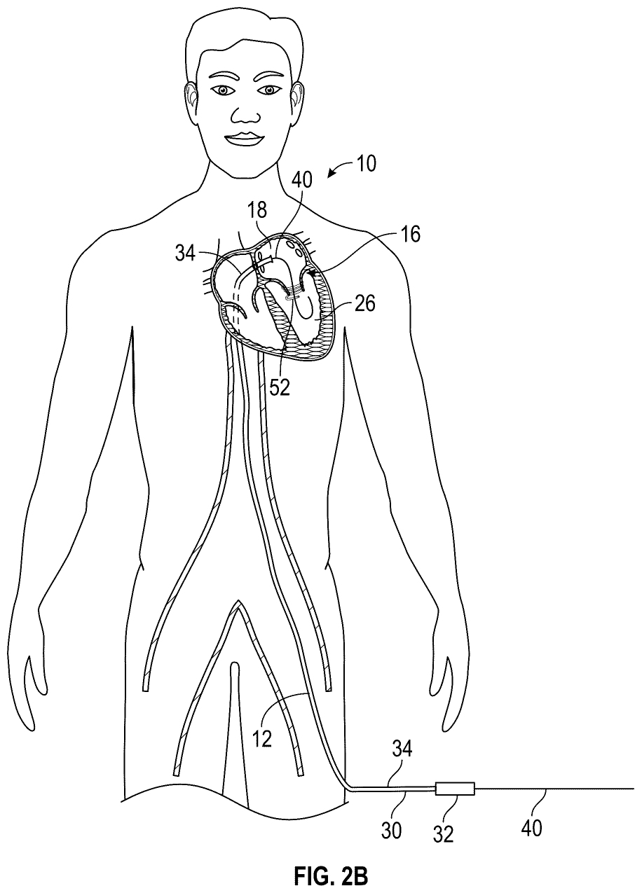

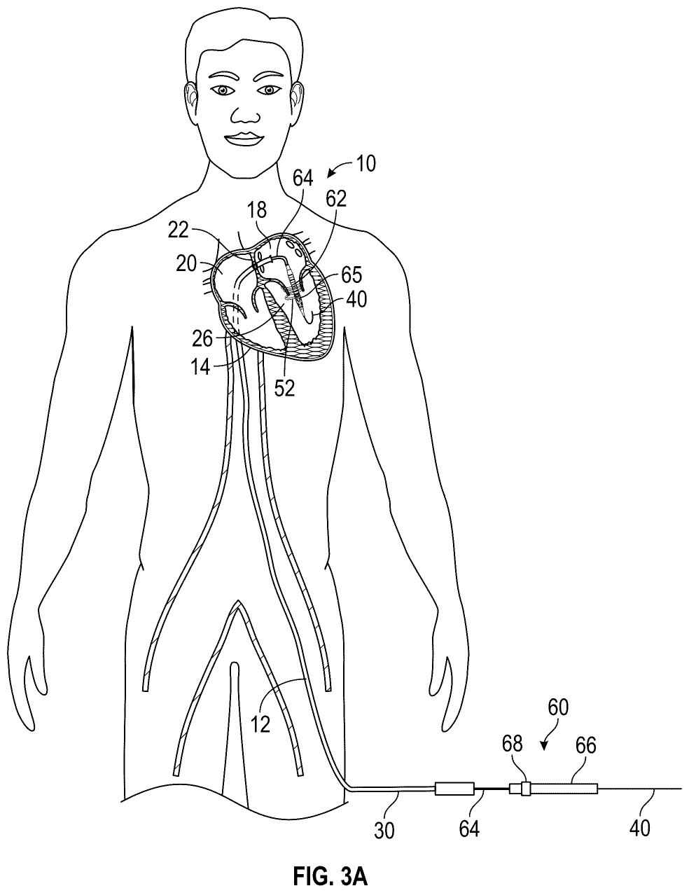

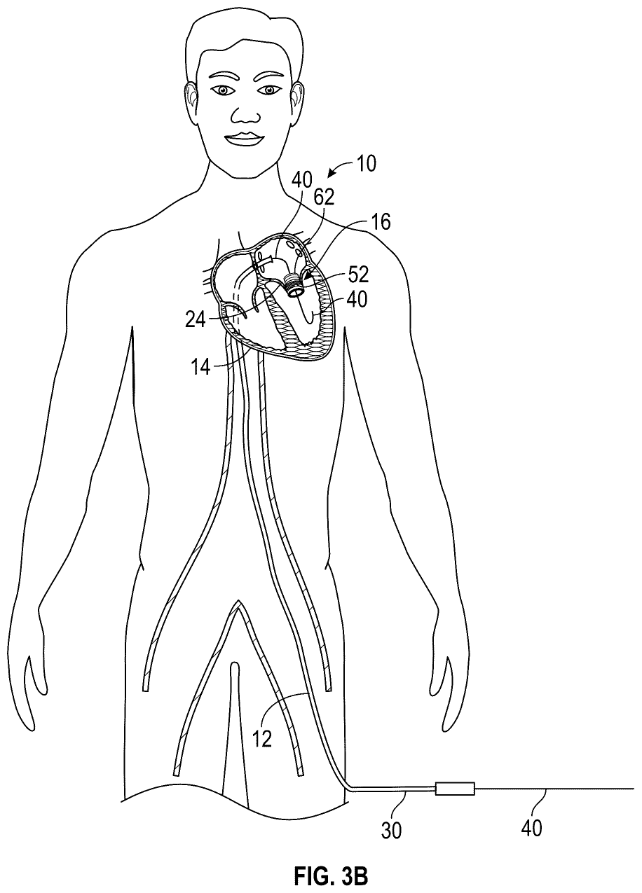

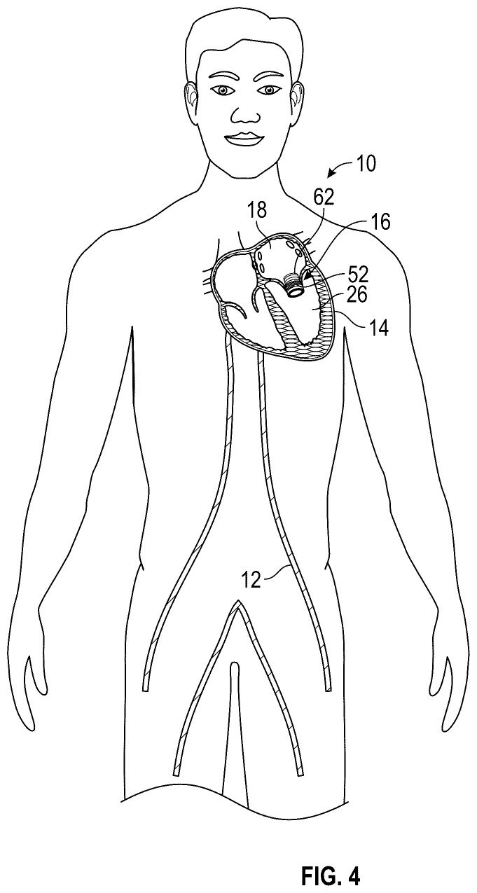

FIGS. 1-4 depict an exemplary transcatheter heart valve replacement procedure (e.g., a mitral valve replacement procedure) which utilizes a docking device 52 and a prosthetic heart valve 62, according to one example. During the procedure, a user first creates a pathway to a patient's native heart valve using a guide catheter 30 (FIG. 1). The user then delivers and implants the docking device 52 at the patient's native heart valve using a docking device delivery apparatus 50 (FIG. 2A) and then removes the docking device delivery apparatus 50 from the patient 10 after implanting the docking device 52 (FIG. 2B). The user then implants the prosthetic heart valve 62 within the implanted docking device 52 using a prosthetic valve delivery apparatus 60 (FIG. 3A). Thereafter, the user removes the prosthetic valve delivery apparatus 60 from the patient 10 (FIG. 3B), as well as the guide catheter 30 (FIG. 4).

Specifically, FIG. 1 depicts a first stage in a mitral valve replacement procedure, according to one example, where the guide catheter 30 and a guidewire 40 are inserted into a blood vessel 12 of a patient 10 and navigated through the blood vessel 12, into a heart 14 of the patient 10, and toward the native mitral valve 16. Together, the guide catheter 30 and the guidewire 40 can provide a path for the docking device delivery apparatus 50 and the prosthetic valve delivery apparatus 60 to be navigated through and along, to the implantation site (the native mitral valve 16 or native mitral valve annulus).

Initially, the user may first make an incision in the patient's body to access the blood vessel 12. For example, in the example illustrated in FIG. 1, the user may make an incision in the patient's groin to access a femoral vein. Thus, in such examples, the blood vessel 12 may be a femoral vein.

After making the incision at the blood vessel 12, the user may insert the guide catheter 30, the guidewire 40, and/or additional devices (such as an introducer device or transseptal puncture device) through the incision and into the blood vessel 12. The guide catheter 30 (which can also be referred to as an “introducer device”, “introducer”, or “guide sheath”) is configured to facilitate the percutaneous introduction of various implant delivery devices (e.g., the docking device delivery apparatus 50 and the prosthetic valve delivery apparatus 60) into and through the blood vessel 12 and may extend through the blood vessel 12 and into the heart 14 but may stop short of the native mitral valve 16. The guide catheter 30 can comprise a handle 32 and a shaft 34 extending distally from the handle 32. The shaft 34 can extend through the blood vessel 12 and into the heart 14 while the handle 32 remains outside the body of the patient 10 and can be operated by the user in order to manipulate the shaft 34 (FIG. 1).

The guidewire 40 is configured to guide the delivery apparatuses (e.g., the guide catheter 30, the docking device delivery apparatus 50, the prosthetic valve delivery apparatus 60, additional catheters, or the like) and their associated devices (e.g., docking device, prosthetic heart valve, and the like) to the implantation site within the heart 14, and thus may extend all the way through the blood vessel 12 and into a left atrium 18 of the heart 14 (and in some examples, through the native mitral valve 16 and into a left ventricle of the heart 14) (FIG. 1).

In some instances, a transseptal puncture device or catheter can be used to initially access the left atrium 18, prior to inserting the guidewire 40 and the guide catheter 30. For example, after making the incision to the blood vessel 12, the user may insert a transseptal puncture device through the incision and into the blood vessel 12. The user may guide the transseptal puncture device through the blood vessel 12 and into the heart 14 (e.g., through the femoral vein and into the right atrium 20). The user can then make a small incision in an atrial septum 22 of the heart 14 to allow access to the left atrium 18 from the right atrium 20. The user can then insert and advance the guidewire 40 through the transseptal puncture device within the blood vessel 12 and through the incision in the atrial septum 22 into the left atrium 18. Once the guidewire 40 is positioned within the left atrium 18 and/or the left ventricle 26, the transseptal puncture device can be removed from the patient 10. The user can then insert the guide catheter 30 into the blood vessel 12 and advance the guide catheter 30 into the left atrium 18 over the guidewire 40 (FIG. 1).

In some instances, an introducer device can be inserted through a lumen of the guide catheter 30 prior to inserting the guide catheter 30 into the blood vessel 12. In some instances, the introducer device can include a tapered end that extends out a distal tip of the guide catheter 30 and that is configured to guide the guide catheter 30 into the left atrium 18 over the guidewire 40. Additionally, in some instances the introducer device can include a proximal end portion that extends out a proximal end of the guide catheter 30. Once the guide catheter 30 reaches the left atrium 18, the user can remove the introducer device from inside the guide catheter 30 and the patient 10. Thus, only the guide catheter 30 and the guidewire 40 remain inside the patient 10. The guide catheter 30 is then in position to receive an implant delivery apparatus and help guide it to the left atrium 18, as described further below.

FIG. 2A depicts a second stage in the exemplary mitral valve replacement procedure where a docking device 52 is being implanted at the native mitral valve 16 of the heart 14 of the patient 10 using a docking device delivery apparatus 50 (which may also be referred to as an “implant catheter” and/or a “docking device delivery device”).

In general, the docking device delivery apparatus 50 comprises a delivery shaft 54, a handle 56, and a pusher assembly 58. The delivery shaft 54 is configured to be advanced through the patient's vasculature (blood vessel 12) and to the implantation site (e.g., native mitral valve 16) by the user and may be configured to retain the docking device 52 in a distal end portion 53 of the delivery shaft 54. In some examples, the distal end portion 53 of the delivery shaft 54 retains the docking device 52 therein in a straightened delivery configuration.

The handle 56 of the docking device delivery apparatus 50 is configured to be gripped and/or otherwise held by the user, outside the body of the patient 10, to advance the delivery shaft 54 through the patient's vasculature (e.g., blood vessel 12).

In some examples, the handle 56 can comprise one or more articulation members 57 (or rotatable knobs) that are configured to aid in navigating the delivery shaft 54 through the blood vessel 12. For example, the one or more articulation members 57 can comprise one or more of knobs, buttons, wheels, and/or other types of physically adjustable control members that are configured to be adjusted by the user to flex, bend, twist, turn, and/or otherwise articulate a distal end portion 53 of the delivery shaft 54 to aid in navigating the delivery shaft 54 through the blood vessel 12 and within the heart 14.

The pusher assembly 58 can be configured to deploy and/or implant the docking device 52 at the implantation site (e.g., the native mitral valve 16). For example, the pusher assembly 58 is configured to be adjusted by the user to push the docking device 52 out of the distal end portion 53 of the delivery shaft 54. A shaft of the pusher assembly 58 can extend through the delivery shaft 54 and can be disposed adjacent to the docking device 52 within the delivery shaft 54. In some examples, the docking device 52 can be releasably coupled to the shaft of the pusher assembly 58 via a connection mechanism of the docking device delivery apparatus 50 such that the docking device 52 can be released after being deployed at the native mitral valve 16.

Further details of the docking device delivery apparatus and its variants are described in International Patent Application Publication No. WO2020/247907, which is incorporated by reference herein in its entirety.

Referring again to FIG. 2A, after the guide catheter 30 is positioned within the left atrium 18, the user may insert the docking device delivery apparatus 50 (e.g., the delivery shaft 54) into the patient 10 by advancing the delivery shaft 54 of the docking device delivery apparatus 50 through the guide catheter 30 and over the guidewire 40. In some examples, the guidewire 40 can be at least partially retracted away from the left atrium 18 and into the guide catheter 30. The user may then continue to advance the delivery shaft 54 of the docking device delivery apparatus 50 through the blood vessel 12 along the guidewire 40 until the delivery shaft 54 reaches the left atrium 18, as illustrated in FIG. 2A. Specifically, the user may advance the delivery shaft 54 of the docking device delivery apparatus 50 by gripping and exerting a force on (e.g., pushing) the handle 56 of the docking device delivery apparatus 50 toward the patient 10. While advancing the delivery shaft 54 through the blood vessel 12 and the heart 14, the user may adjust the one or more articulation members 57 of the handle 56 to navigate the various turns, corners, constrictions, and/or other obstacles in the blood vessel 12 and the heart 14.

Once the delivery shaft 54 reaches the left atrium 18 and extends out of a distal end of the guide catheter 30, the user can position the distal end portion 53 of the delivery shaft 54 at and/or near the posteromedial commissure of the native mitral valve 16 using the handle 56 (e.g., the articulation members 57). The user may then push the docking device 52 out of the distal end portion 53 of the delivery shaft 54 with the shaft of the pusher assembly 58 to deploy and/or implant the docking device 52 within the annulus of the native mitral valve 16.

In some examples, the docking device 52 may be constructed from, formed of, and/or comprise a shape memory material, and as such, may return to its original, pre-formed shape when it exits the delivery shaft 54 and is no longer constrained by the delivery shaft 54. As one example, the docking device 52 may originally be formed as a coil, and thus may wrap around leaflets 24 of the native mitral valve 16 as it exits the delivery shaft 54 and returns to its original coiled configuration.

After pushing a ventricular portion of the docking device 52 (e.g., the portion of the docking device 52 shown in FIG. 2A that is configured to be positioned within a left ventricle 26 and/or on the ventricular side of the native mitral valve 16), the user may then deploy the remaining portion of the docking device 52 (e.g., an atrial portion of the docking device 52) from the delivery shaft 54 within the left atrium 18 by retracting the delivery shaft 54 away from the posteromedial commissure of the native mitral valve 16.

After deploying and implanting the docking device 52 at the native mitral valve 16, the user may disconnect the docking device delivery apparatus 50 from the docking device 52. Once the docking device 52 is disconnected from the docking device delivery apparatus 50, the user may retract the docking device delivery apparatus 50 out of the blood vessel 12 and away from the patient 10 so that the user can deliver and implant a prosthetic heart valve 62 within the implanted docking device 52 at the native mitral valve 16.

FIG. 2B depicts this third stage in the mitral valve replacement procedure, where the docking device 52 has been fully deployed and implanted at the native mitral valve 16 and the docking device delivery apparatus 50 (including the delivery shaft 54) has been removed from the patient 10 such that only the guidewire 40 and the guide catheter 30 remain inside the patient 10. In some examples, after removing the docking device delivery apparatus, the guidewire 40 can be advanced out of the guide catheter 30, through the implanted docking device 52 at the native mitral valve 16, and into the left ventricle 26 (FIG. 2A). As such, the guidewire 40 can help to guide the prosthetic valve delivery apparatus 60 through the annulus of the native mitral valve 16 and at least partially into the left ventricle 26.

As illustrated in FIG. 2B, the docking device 52 can comprise a plurality of turns (or revolutions) of a coil that wrap around the leaflets 24 of the native mitral valve 16 (within the left ventricle 26). The implanted docking device 52 has a more cylindrical shape than the annulus of the native mitral valve 16, thereby providing a geometry that more closely matches the shape or profile of the prosthetic heart valve to be implanted. As a result, the docking device 52 can provide a tighter fit, and thus a better seal, between the prosthetic heart valve and the native mitral valve 16, as described further below.

FIG. 3A depicts a fourth stage in the mitral valve replacement procedure where the user is delivering and/or implanting a prosthetic heart valve 62 (which can also be referred to herein as a “transcatheter prosthetic heart valve” or “THV” for short, “replacement heart valve,” “prosthetic mitral valve,” and/or “prosthetic valve”) within the docking device 52 using a prosthetic valve delivery apparatus 60.

As shown in FIG. 3A, the prosthetic valve delivery apparatus 60 can comprise a delivery shaft 64 and a handle 66, the delivery shaft 64 extending distally from the handle 66. The delivery shaft 64 is configured to extend into the patient's vasculature to deliver, implant, expand, and/or otherwise deploy the prosthetic heart valve 62 within the coiled docking device 52 at the native mitral valve 16. The handle 66 is configured to be gripped and/or otherwise held by the user to advance the delivery shaft 64 through the patient's vasculature.

In some examples, the handle 66 can comprise one or more articulation members 68 that are configured to aid in navigating the delivery shaft 64 through the blood vessel 12 and the heart 14. Specifically, the articulation member(s) 68 can comprise one or more of knobs, buttons, wheels, and/or other types of physically adjustable control members that are configured to be adjusted by the user to flex, bend, twist, turn, and/or otherwise articulate a distal end portion of the delivery shaft 64 to aid in navigating the delivery shaft 64 through the blood vessel 12 and into the left atrium 18 and left ventricle 26 of the heart 14.

In some examples, the prosthetic valve delivery apparatus 60 can include an expansion mechanism 65 that is configured to radially expand and deploy the prosthetic heart valve 62 at the implantation site. In some instances, as shown in FIG. 3A, the expansion mechanism 65 can comprise an inflatable balloon that is configured to be inflated to radially expand the prosthetic heart valve 62 within the docking device 52. The inflatable balloon can be coupled to the distal end portion of the delivery shaft 64.

In some examples, the prosthetic heart valve 62 can be self-expanding and can be configured to radially expand on its own upon removable of a sheath or capsule covering the radially compressed prosthetic heart valve 62 on the distal end portion of the delivery shaft 64. In some examples, the prosthetic heart valve 62 can be mechanically expandable and the prosthetic valve delivery apparatus 60 can include one or more mechanical actuators (e.g., the expansion mechanism) configured to radially expand the prosthetic heart valve 62.

As shown in FIG. 3A, the prosthetic heart valve 62 is mounted around the expansion mechanism 65 (the inflatable balloon) on the distal end portion of the delivery shaft 64, in a radially compressed configuration.

To navigate the distal end portion of the delivery shaft 64 to the implantation site, the user can insert the prosthetic valve delivery apparatus 60 (the delivery shaft 64) into the patient 10 through the guide catheter 30 and over the guidewire 40. The user can continue to advance the prosthetic valve delivery apparatus 60 along the guidewire 40 (through the blood vessel 12) until the distal end portion of the delivery shaft 64 reaches the native mitral valve 16, as illustrated in FIG. 3A. More specifically, the user can advance the delivery shaft 64 of the prosthetic valve delivery apparatus 60 by gripping and exerting a force on (e.g., pushing) the handle 66. While advancing the delivery shaft 64 through the blood vessel 12 and the heart 14, the user can adjust the one or more articulation members 68 of the handle 66 to navigate the various turns, corners, constrictions, and/or other obstacles in the blood vessel 12 and heart 14.

The user can advance the delivery shaft 64 along the guidewire 40 until the radially compressed prosthetic heart valve 62 mounted around the distal end portion of the delivery shaft 64 is positioned within the docking device 52 and the native mitral valve 16. In some examples, as shown in FIG. 3A, a distal end of the delivery shaft 64 and a least a portion of the radially compressed prosthetic heart valve 62 can be positioned within the left ventricle 26.

Once the radially compressed prosthetic heart valve 62 is appropriately positioned within the docking device 52 (FIG. 3A), the user can manipulate one or more actuation mechanisms of the handle 66 of the prosthetic valve delivery apparatus 60 to actuate the expansion mechanism 65 (e.g., inflate the inflatable balloon), thereby radially expanding the prosthetic heart valve 62 within the docking device 52.

FIG. 3B shows a fifth stage in the mitral valve replacement procedure where the prosthetic heart valve 62 in its radially expanded configuration and implanted within the docking device 52 in the native mitral valve 16. As shown in FIG. 3B, the prosthetic heart valve 62 is received and retained within the coiled docking device 52. Thus, the docking device 52 aids in anchoring the prosthetic heart valve 62 within the native mitral valve 16. The docking device 52 can enable better sealing between the prosthetic heart valve 62 and the leaflets 24 of the native mitral valve 16 to reduce paravalvular leakage (PVL) around the prosthetic heart valve 62.

As also shown in FIG. 3B, after the prosthetic heart valve 62 has been fully deployed and implanted within the docking device 52 at the native mitral valve 16, the prosthetic valve delivery apparatus 60 (including the delivery shaft 64) is removed from the patient 10 such that only the guidewire 40 and the guide catheter 30 remain inside the patient 10.

FIG. 4 depicts a sixth stage in the mitral valve replacement procedure, where the guidewire 40 and the guide catheter 30 have been removed from the patient 10.

Although FIGS. 1-4 specifically depict a mitral valve replacement procedure, it should be appreciated that the same and/or similar procedure may be utilized to replace other heart valves (e.g., tricuspid, pulmonary, and/or aortic valves). Further, the same and/or similar delivery apparatuses (e.g., docking device delivery apparatus 50, prosthetic valve delivery apparatus 60, guide catheter 30, and/or guidewire 40), docking devices (e.g., docking device 52), replacement heart valves (e.g., prosthetic heart valve 62), and/or components thereof may be utilized for replacing other heart valves.

For example, when replacing a native tricuspid valve, the user may also access the right atrium 20 via a femoral vein but may not need to cross the atrial septum 22 into the left atrium 18. Instead, the user may leave the guidewire 40 in the right atrium 20 and perform the same and/or similar docking device implantation process at the tricuspid valve. Specifically, the user may push the docking device 52 out of the delivery shaft 54 around the ventricular side of the tricuspid valve leaflets, release the remaining portion of the docking device 52 from the delivery shaft 54 within the right atrium 20, and then remove the delivery shaft 54 of the docking device delivery apparatus 50 from the patient 10. The user may then advance the guidewire 40 through the tricuspid valve into the right ventricle and perform the same and/or similar prosthetic heart valve implantation process at the tricuspid valve, within the docking device 52. Specifically, the user may advance the delivery shaft 64 of the prosthetic valve delivery apparatus 60 through the patient's vasculature along the guidewire 40 until the prosthetic heart valve 62 is positioned/disposed within the docking device 52 and the tricuspid valve. The user may then expand the prosthetic heart valve 62 within the docking device 52 before removing the prosthetic valve delivery apparatus 60 from the patient 10. In another example, the user may perform the same and/or similar process to replace the aortic valve but may access the aortic valve from the outflow side of the aortic valve via a femoral artery.

Further, although FIGS. 1-4 depict a mitral valve replacement procedure that accesses the native mitral valve 16 from the left atrium 18 via the right atrium 20 and femoral vein, it should be appreciated that the native mitral valve 16 may alternatively be accessed from the left ventricle 26. For example, the user may access the native mitral valve 16 from the left ventricle 26 via the aortic valve by advancing one or more delivery apparatuses through an artery to the aortic valve, and then through the aortic valve into the left ventricle 26.

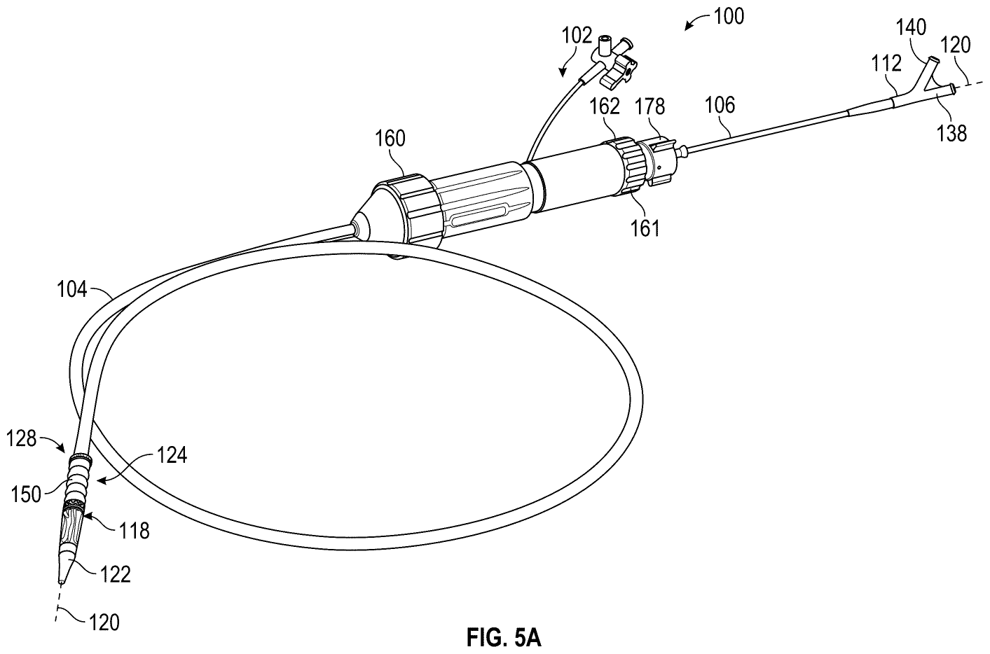

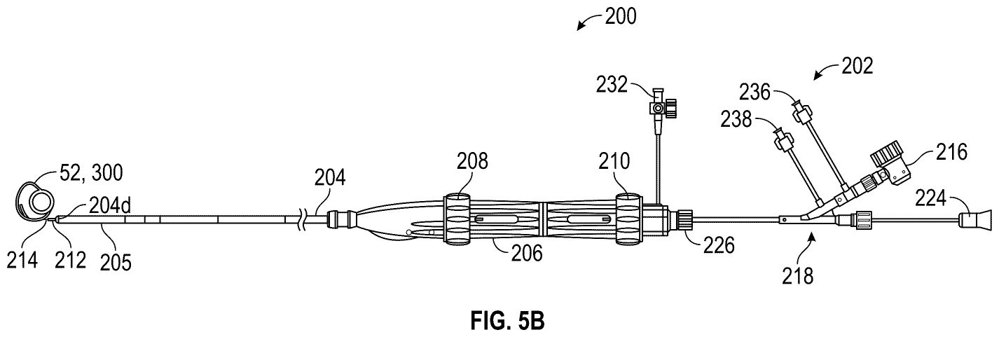

FIG. 5A illustrates an exemplary prosthetic heart valve delivery apparatus 100 (which can also be referred to here as an “implant catheter”) that can be used in lieu of the prosthetic valve delivery apparatus 60 of FIG. 3A to implant an expandable prosthetic heart valve, such as the prosthetic valves 62, 150, or 400 described herein. In some examples, the delivery apparatus 100 is specifically adapted for use in introducing a prosthetic heart valve into a heart.

The delivery apparatus 100 in the illustrated example of FIG. 5A is a balloon catheter comprising a handle 102 and a steerable, outer shaft 104 extending distally from the handle 102. The delivery apparatus 100 can further comprise an intermediate shaft 106 (which also may be referred to as a balloon shaft) that extends proximally from the handle 102 and distally from the handle 102, the portion extending distally from the handle 102 also extending coaxially through the outer shaft 104. In some examples, the delivery apparatus 100 can further comprise an inner shaft extending distally from the handle 102 coaxially through the intermediate shaft 106 and the outer shaft 104 and proximally from the handle 102 coaxially through the intermediate shaft.

The outer shaft 104 and the intermediate shaft 106 can be configured to translate (e.g., move) longitudinally, along a central longitudinal axis 120 of the delivery apparatus 100, relative to one another to facilitate delivery and positioning of a prosthetic valve at an implantation site in a patient's body.

The intermediate shaft 106 can include a proximal end portion that extends proximally from a proximal end of the handle 102, to an adaptor 112. The adaptor 112 can include a first port 138 configured to receive a guidewire therethrough and a second port 140 configured to receive fluid (e.g., inflation fluid) from a fluid source. The second port 140 can be fluidly coupled to an inner lumen of the intermediate shaft 106.

In some examples, the intermediate shaft 106 can further include a distal end portion that extends distally beyond a distal end of the outer shaft 104 when a distal end of the outer shaft 104 is positioned away from an inflatable balloon 118 of the delivery apparatus 100. A distal end portion of the inner shaft can extend distally beyond the distal end portion of the intermediate shaft 106 toward or to a nose cone 122 at a distal end of the delivery apparatus 100.

In some examples, a distal end of the balloon 118 can be coupled to a distal end of the delivery apparatus 100, such as to the nose cone 122 (as shown in FIG. 5A), or to an alternate component at the distal end of the delivery apparatus 100 (e.g., a distal shoulder). An intermediate portion of the balloon 118 can overlay a valve mounting portion 124 of a distal end portion of the delivery apparatus 100 and a distal end portion of the balloon 118 (shown in FIG. 5A) can overly a distal shoulder of the delivery apparatus 100. As shown in FIG. 5A, a prosthetic heart valve 150 can be mounted around the balloon 118, at the valve mounting portion 124 of the delivery apparatus 100, in a radially compressed state. The prosthetic heart valve 150 can be configured to be radially expanded by inflation of the balloon 118 at a native valve annulus, as described above with reference to FIGS. 3A and 3B.

A balloon shoulder assembly of the delivery apparatus 100, which includes the distal shoulder, is configured to maintain the prosthetic heart valve 150 (or other medical device) at a fixed position on the balloon 118 during delivery through the patient's vasculature.

The outer shaft 104 can include a distal tip portion 128 mounted on its distal end. In some examples, the outer shaft 104 and the intermediate shaft 106 can be translated axially relative to one another to position the distal tip portion 128 adjacent to a proximal end of the valve mounting portion 124, when the prosthetic valve 150 is mounted in the radially compressed state on the valve mounting portion 124 (as shown in FIG. 5A) and during delivery of the prosthetic valve to the target implantation site. As such, the distal tip portion 128 can be configured to resist movement of the prosthetic valve 150 relative to the balloon 118 proximally, in the axial direction, relative to the balloon 118, when the distal tip portion 128 is arranged adjacent to a proximal side of the valve mounting portion 124.

An annular space can be defined between an outer surface of the inner shaft and an inner surface of the intermediate shaft 106 and can be configured to receive fluid from a fluid source via the second port 140 of the adaptor 112. The annular space can be fluidly coupled to a fluid passageway formed between the outer surface of the distal end portion of the inner shaft and an inner surface of the balloon 118. As such, fluid from the fluid source can flow to the fluid passageway from the annular space to inflate the balloon 118 and radially expand and deploy the prosthetic valve 150.

An inner lumen of the inner shaft can be configured to receive a guidewire therethrough, for navigating the distal end portion of the delivery apparatus 100 to the target implantation site.

The handle 102 can include a steering mechanism configured to adjust the curvature of the distal end portion of the delivery apparatus 100. In the illustrated example, for example, the handle 102 includes an adjustment member, such as the illustrated rotatable knob 160, which in turn is operatively coupled to the proximal end portion of a pull wire. The pull wire can extend distally from the handle 102 through the outer shaft 104 and has a distal end portion affixed to the outer shaft 104 at or near the distal end of the outer shaft 104. Rotating the knob 160 can increase or decrease the tension in the pull wire, thereby adjusting the curvature of the distal end portion of the delivery apparatus 100. Further details on steering or flex mechanisms for the delivery apparatus can be found in U.S. Pat. No. 9,339,384, which is incorporated by reference herein.

The handle 102 can further include an adjustment mechanism 161 including an adjustment member, such as the illustrated rotatable knob 162, and an associated locking mechanism including another adjustment member, configured as a rotatable knob 178. The adjustment mechanism 161 is configured to adjust the axial position of the intermediate shaft 106 relative to the outer shaft 104 (e.g., for fine positioning at the implantation site). Additional features of the delivery apparatus 100 that can be utilized with the prosthetic valve delivery apparatus, systems, and methods disclosed herein are described in U.S. Provisional Patent Application 63/366,897 filed on Jun. 23, 2022, which is incorporated by reference herein.

FIG. 5B illustrates a docking device delivery apparatus 200 that can be configured to implant a docking device, such as the docking devices 52 or 300 described herein or other docking devices, and that can be used in lieu of the docking device delivery apparatus 50 of FIG. 2A. In some examples, the docking device delivery apparatus 200 is specifically adapted for use in introducing a docking device into a heart. The docking device delivery apparatus can also be referred to as a “dock delivery catheter” or “dock delivery system.”

As shown, the delivery apparatus 200 can include a handle assembly 202 and a delivery sheath 204 (also referred to as the “delivery shaft” or “outer shaft” or “outer sheath”) extending distally from the handle assembly 202. The handle assembly 202 can include a handle 206 including one or more knobs, buttons, wheels, and/or other means for controlling and/or actuating one or more components of the delivery apparatus 200. For example, in some examples, as shown in FIG. 5B, the handle 206 can include knobs 208 and 210 which can be configured to steer or control flexing of components of the delivery apparatus 200.

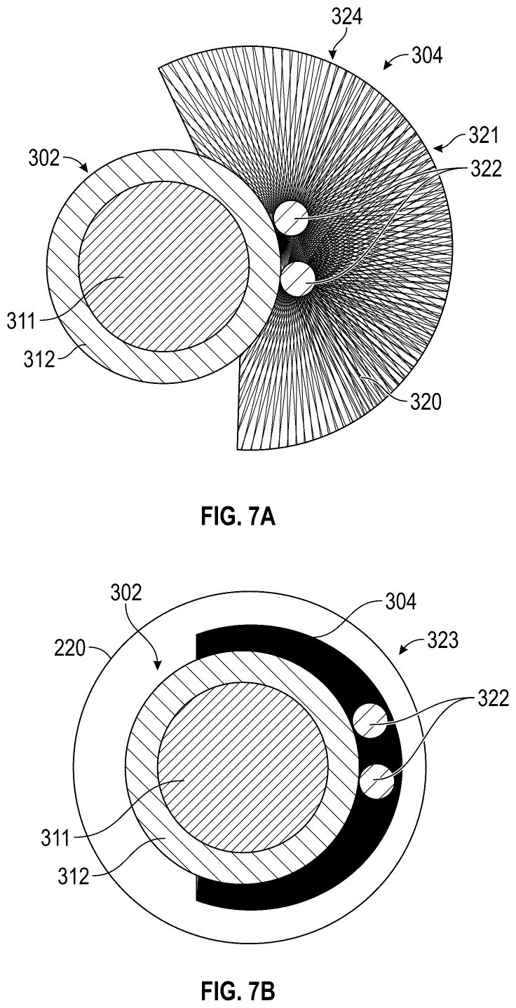

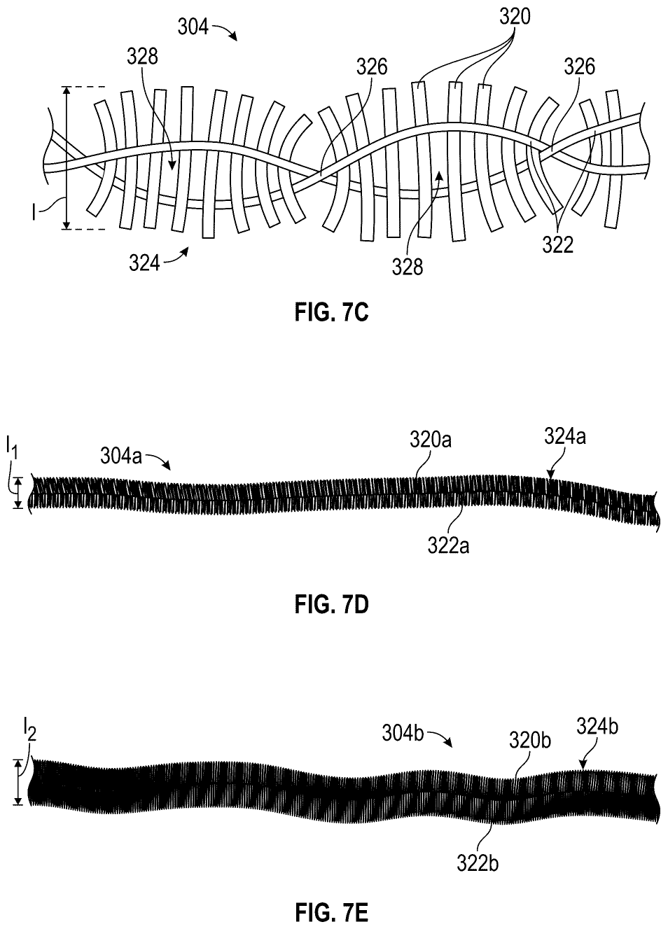

In some examples, the delivery apparatus 200 can also include a pusher shaft 212 and/or a sleeve shaft (not shown), both of which can extend through an inner lumen of the delivery sheath 204 and have respective proximal end portions extending into the handle assembly 202. A distal end portion (also referred to as “distal section”) of the sleeve shaft can include a lubricous dock sleeve configured to cover or surround the docking device 52, 300. For example, as shown in FIG. 7B and further discussed below, a docking device 300 can be retained or disposed inside of a dock sleeve 220. In some examples, the docking device 300 within the dock sleeve 220 can be further retained by or disposed within a distal end portion 205 of the delivery sheath 204, when navigating through a patient's vasculature.

Additionally, the distal end portion 205 of the delivery sheath 204 can be configured to be steerable. In one example, by rotating a knob (e.g., 208 or 210) on the handle 206, a curvature of the distal end portion 205 can be adjusted so that the distal end portion 205 of the delivery sheath 204 can be oriented in a desired angle. For example, to implant the docking device 52, 300 at the native mitral valve location, the distal end portion 205 of the delivery sheath 204 can be steered in the left atrium so that the dock sleeve 220 and the docking device 52, 300 retained therein can extend through the native mitral valve annulus at a location adjacent the posteromedial commissure.

In some examples, the pusher shaft 212 and the sleeve shaft (not shown) can be coaxial with one another, at least within the delivery sheath 204. In addition, the delivery sheath 204 can be configured to be axially movable relative to the sleeve shaft and the pusher shaft 212. As described further below, a distal end of the pusher shaft 212 can be inserted into a lumen of the sleeve shaft and press against the proximal end of the docking device 52, 300 retained inside the dock sleeve 220.

After reaching a target implantation site, the docking device 52, 300 can be deployed from the delivery sheath 204 by manipulating the pusher shaft 212 and sleeve shaft using a hub assembly 218, as described further below. For example, by pushing the pusher shaft in the distal direction while holding the delivery sheath 204 in place or retracting the delivery sheath 204 in the proximal direction while holding the pusher shaft in place, or pushing the pusher shaft 212 in the distal direction while simultaneously retracting the delivery sheath 204 in the proximal direction, the docking device 100 can be pushed out of a distal end 204d of the delivery sheath 204, thus changing from the delivery configuration to the deployed configuration. In some examples, the pusher shaft 212 and the sleeve shaft can be actuated independently of each other.

In some examples, when deploying the docking device 52, 300 from the delivery sheath 204, the pusher shaft 212 and the sleeve shaft can be configured to move together with the docking device 52, 300 in the axial direction. For example, actuation of the pusher shaft 212, to push against the docking device 52, 300 and move it out of the delivery sheath 204 can also cause the sleeve shaft to move along with the pusher shaft 212 and the docking device 52, 300.

As such, the docking device 52, 300 can remain covered by the dock sleeve 220 of the sleeve shaft during the procedure of pushing the docking device 52, 300 into position at the target implantation site via the pusher shaft 212. Thus, in some examples, when the docking device 52, 300 is initially deployed at the target implantation site, the lubricous dock sleeve 220 can facilitate the covered docking device 52, 300 encircling the native anatomy.

During delivery, the docking device 52, 300 can be coupled to the delivery apparatus 200 via a release suture 214 (or other retrieval line comprising a string, yarn, or other material that can be configured to be tied around the docking device 52, 300 and cut for removal) that extends through the pusher shaft 212. In one specific example, the release suture 214 can extend through the delivery apparatus 200, e.g., through an inner lumen of the pusher shaft 212, to a suture lock assembly 216 of the delivery apparatus 200.

The handle assembly 202 can further include a hub assembly 218 to which the suture lock assembly 216 and a sleeve handle 224 are attached. The hub assembly 218 can be configured to independently control the pusher shaft 212 and the sleeve shaft while the sleeve handle 224 can control an axial position of the sleeve shaft relative to the pusher shaft 212. In this way, operation of the various components of the handle assembly 202 can actuate and control operation of the components arranged within the delivery sheath 204. In some examples, the hub assembly 218 can be coupled to the handle 206 via a connector 226.

The handle assembly 202 can further include one or more flushing ports (e.g., three flushing ports 232, 236, 238 are shown in FIG. 5B) to supply flush fluid to one or more lumens arranged within the delivery apparatus 200 (e.g., annular lumens arranged between coaxial components of the delivery apparatus 200).

Further details on delivery apparatus/catheters/systems (including some examples of the handle assembly) that are configured to deliver a docking device to a target implantation site can be found in U.S. Patent Application Publication Nos. 2018/0318079 and 2018/0263764, and PCT Patent Application No. US2021/056150, which are each incorporated by reference herein.

In some examples, one or more of the foregoing delivery techniques and methods are carried out in a simulation procedure, which are not conducted on a living human body. For example, the methods and techniques can be performed on a model anatomical system, in a cadaver, or in an animal.

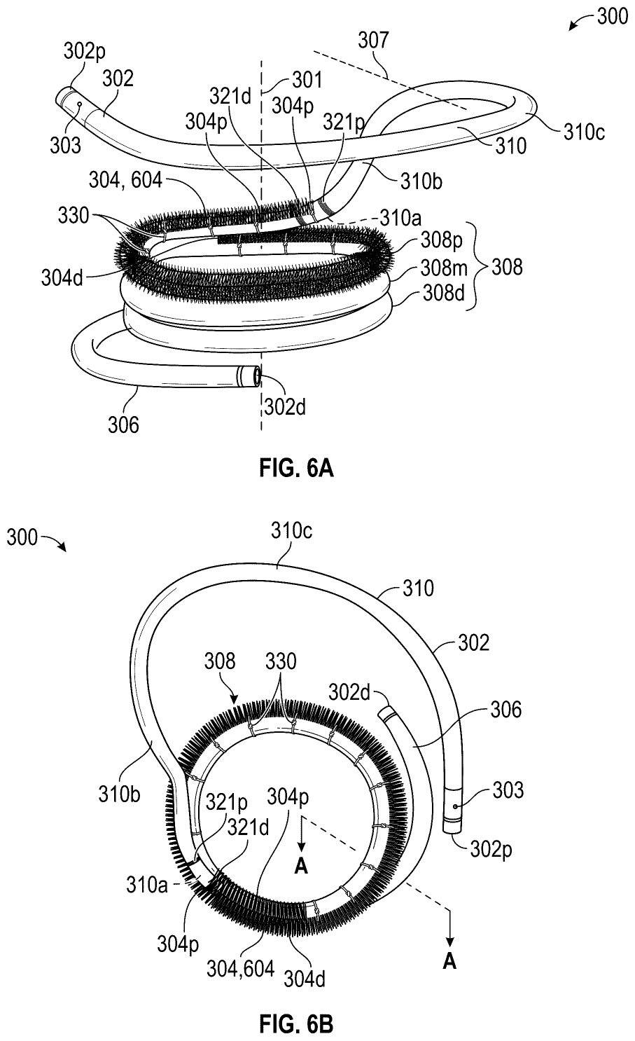

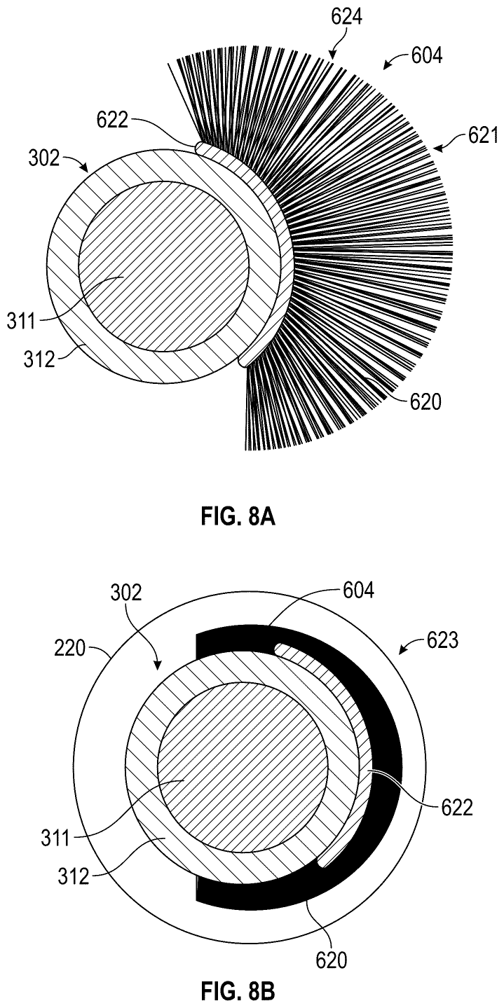

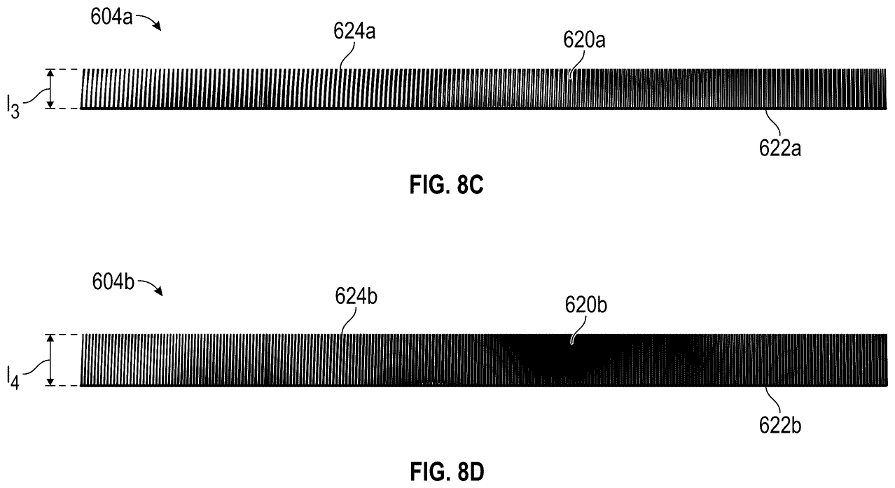

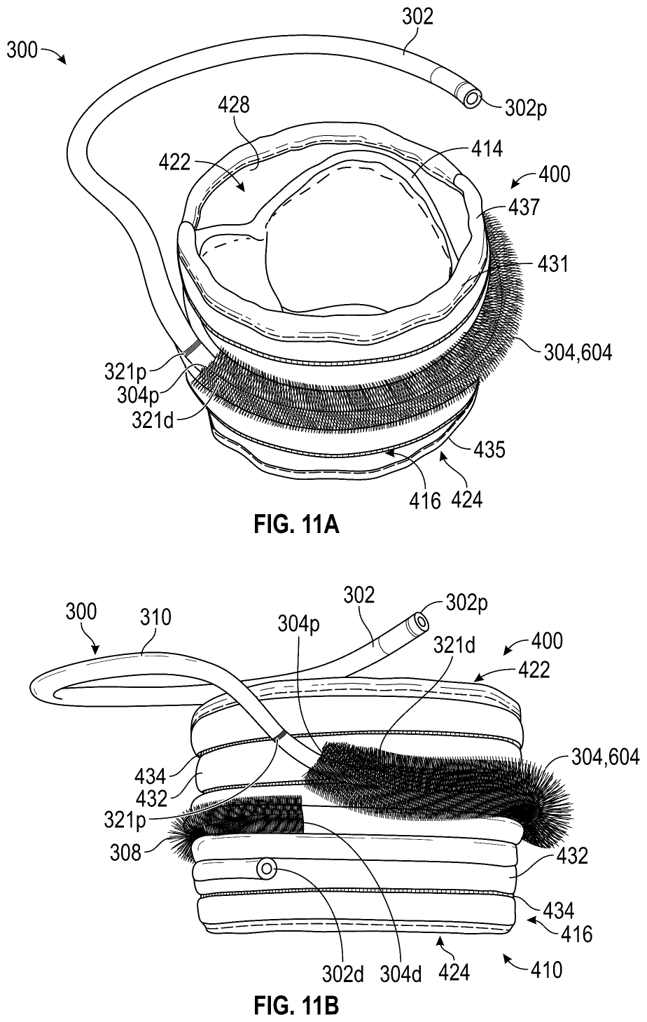

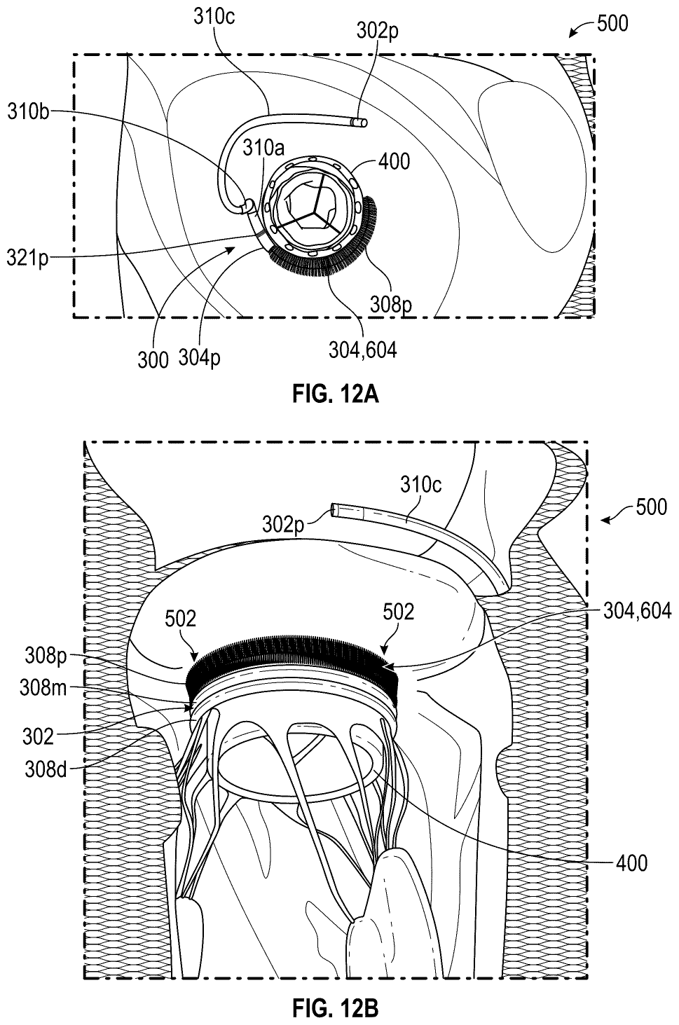

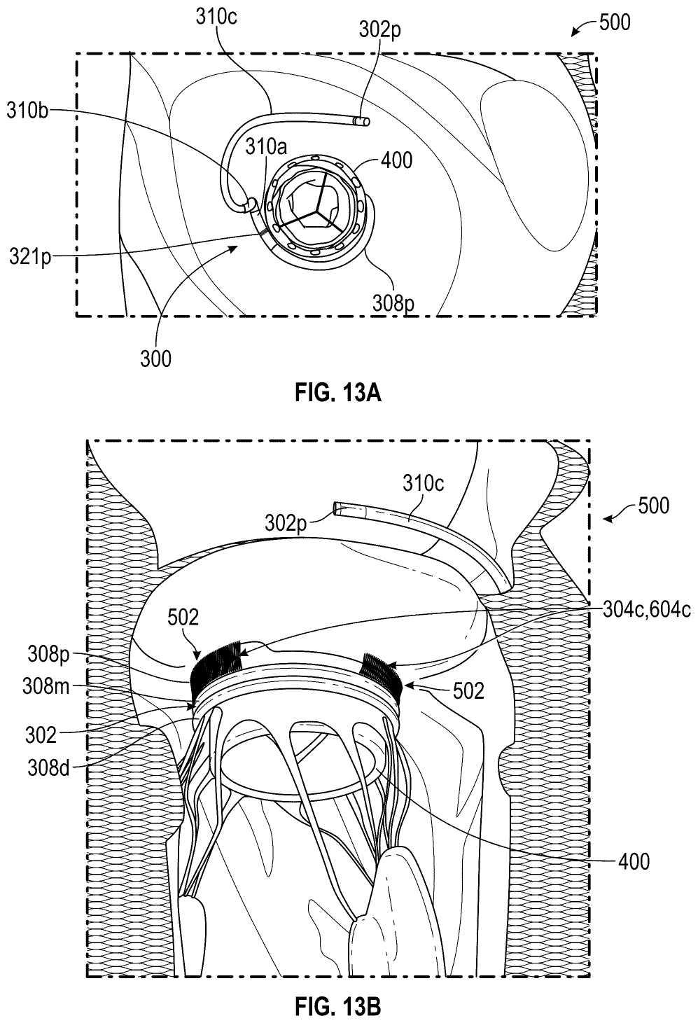

Turning now to FIGS. 6A-13B exemplary docking devices, prosthetic valves, and methods of implantation thereof are illustrated and described. Specifically, FIGS. 6A and 6B show a docking device 300, according to one example. Exemplary PVL guards or guard members 304, 604 that can be utilized with the docking device 300 are illustrated in FIGS. 7A-8D. As depicted in FIGS. 11A-13B, the docking device 300 can be configured to receive and secure and/or retain a prosthetic valve 400 within the docking device, thereby securing the prosthetic valve at the native valve annulus. The docking device 300 and the prosthetic valve 400 can be implanted, for example, within a native valve annulus of a mitral valve (see, e.g., FIGS. 12A-13B).

Referring to FIGS. 6A and 6B, the docking device 300 can comprise a coil 302 and a guard member 304, 604 covering at least a portion of the coil 302. In some examples, the coil 302 comprises an interior coil member 311 (FIGS. 7A-8B) comprised of a shape memory material (e.g., nickel titanium alloy or “Nitinol”) that can enable the docking device 300 (and the coil member 311) to move from a substantially straight configuration (also referred to as “delivery configuration”) when disposed within a delivery sheath of a delivery apparatus (as described above) to a helical configuration (also referred to as “deployed configuration” or “coiled configuration” as shown in FIGS. 6A-6B) after being removed from the delivery sheath.

The guard member 304, 604 can extend circumferentially relative to a central longitudinal axis 301 of the coil 302. In some examples, the guard member 304, 604 can extend, from a proximal end 304p to a distal end 304d thereof, from 180 degrees to 540 degrees, or from 210 degrees to 360 degrees, or from 250 degrees to 290 degrees, or from 260 degrees to 280 degrees around the coil 302. In other words, in some examples, the guard member 304, 604 can extend circumferentially from about one half of a revolution (e.g., 180 degrees) around the central longitudinal axis 301 to two a full revolutions (e.g., 540 degrees) around the coil 302, including various ranges in between. As used herein, a range (e.g., 180-400 degrees, from 180 degrees to 400 degrees, and between 180 degrees and 400 degrees) includes the endpoints of the range (e.g., 180 degrees and 400 degrees). In one particular example, prior to engagement with a prosthetic valve, the guard member 304, 604 extends circumferentially approximately 390 degrees around the coil 302, such that there is an overlapping region of the guard member 304 between the proximal end 304p and the distal end 304d (corresponding to approximately 30 degrees around the longitudinal axis 301).

The coil 302 has a proximal end 302p and a distal end 302d (which also respectively define the proximal and distal ends of the docking device 300). When disposed within the delivery sheath (e.g., during delivery of the docking device into the vasculature of a patient), the coil member 311 (between the proximal end 302p and distal end 302d) can form a generally straight delivery configuration (i.e., without any coiled or looped portions, but can be flexed or bent) so as to maintain a small radial profile when moving through a patient's vasculature. After being removed from the delivery sheath and deployed at an implant position, the coil member 311 can form the coil 302, thereby moving from the delivery configuration to the helical deployed configuration, which can wrap around native tissue adjacent the implant position. For example, when implanting the docking device 300 at the location of a native valve, the coil 302 can be configured to surround native leaflets of the native valve and the chordae tendineae that connects native leaflets to adjacent papillary muscles, if present (as illustrated in FIGS. 12B and 13B).

The docking device 300 can be releasably coupled to a delivery apparatus. For example, in some examples, the docking device 300 can be coupled to a delivery apparatus (such as the delivery apparatus 50, 200 described above) via a release suture (e.g., the release suture 214 shown in FIG. 5B) that can be configured to be tied to the docking device 300 and subsequently cut for separation of the docking device 300 from the delivery apparatus. In some example, the release suture can be tied to the docking device 300 through an eyelet or eyehole 303 located adjacent the proximal end 302p of the coil. In some examples, the release suture can be tied around a circumferential recess that is located adjacent the proximal end 302p of the coil 302 or can include a different attachment mechanism.

In some examples, the docking device 300 in the deployed or coiled configuration can be configured to fitted at the mitral valve (as shown in the examples of FIGS. 12A-13B). In some examples, the docking device can also be shaped and/or adapted for implantation at other native valve positions as well, such as at the tricuspid valve. As described herein, the geometry of the docking device 300 can be configured to engage the native anatomy, which can, for example, provide for increased stability and reduction of relative motion between the docking device 300, the prosthetic valve docked therein, and/or the native anatomy. Reduction of such relative motion can, among other things, prevent material degradation of components of the docking device 300 and/or the prosthetic valve docked therein, and/or prevent damage or trauma to the native tissue.

As shown in FIGS. 6A and 6B, the coil 302 in the deployed configuration can include a leading turn 306 (or “leading coil”), a central region 308, and a stabilization turn 310 (or “stabilization coil”) formed around the central longitudinal axis 301. The central region 308 can possess one or more helical turns having substantially equal inner diameters. The leading turn 306 can extend from a distal end of the central region 308 and can have a diameter greater than the diameter of the central region 308. The stabilization turn 310 can extend from a proximal end of the central region 308 and can have a diameter greater than the diameter of the central region 308.

In some examples, the central region 308 can include a plurality of helical turns, such as a proximal turn 308p in connection with the stabilization turn 310, a distal turn 308d in connection with the leading turn 306, and one or more intermediate turns 308m disposed between the proximal turn 308p and the distal turn 308d. In the example shown in FIG. 6A, there is only one intermediate turn 308m between the proximal turn 308p and the distal turn 308d. In some examples, there are more than one intermediate turns 308m between the proximal turn 308p and the distal turn 308d. Some of the helical turns in the central region 308 can be full turns (i.e., rotating 360 degrees). In some examples, the proximal turn 308p and/or the distal turn 308d can be partial turns (e.g., rotating less than 360 degrees, such as 180 degrees, 270 degrees, etc.).

A size of the docking device 300 can be generally selected based on the size of the desired prosthetic valve to be implanted into the patient. In some examples, the central region 308 can be configured to retain or anchor a radially expandable prosthetic valve (as shown in FIGS. 11A and 11B and described further below). For example, the inner diameter of the helical turns in the central region 308 can be configured to be smaller than an outer diameter of the prosthetic valve when the prosthetic valve is radially expanded so that additional radial force can act between the central region 308 and the prosthetic valve to hold the prosthetic valve in place or otherwise resist movement of the prosthetic valve relative to the native anatomy. As described herein, the helical turns (e.g., 308p, 308m, 308d) in the central region 308 are also referred to herein as “functional turns.”

The stabilization turn 310 can be configured to help stabilize the docking device 300 in the desired position. For example, the radial dimension of the stabilization turn 310 can be significantly larger than the radial dimension of the coil in the central region 308, so that the stabilization turn 310 can flare or extend sufficiently outwardly so as to abut or push against the walls of the circulatory system, thereby improving the ability of the docking device 300 to stay in its desired position prior to the implantation of the prosthetic valve. In some examples, the diameter of stabilization turn 310 is desirably larger than the native annulus, native valve plane, and/or native chamber for better stabilization. In some examples, the stabilization turn 310 can be a full turn (i.e., rotating about 360 degrees). In some examples, the stabilization turn 310 can be a partial turn (e.g., rotating between about 180 degrees and about 270 degrees).

In one particular example, when implanting the docking device 300 at the native mitral valve location, the functional turns in the central region 308 can be disposed substantially in the left ventricle and the stabilization turn 310 can be disposed substantially in the left atrium (see e.g., FIGS. 12A-13B). The stabilization turn 310 can be configured to provide one or more points or regions of contact between the docking device 300 and the left atrial wall, such as at least three points of contact in the left atrium or complete contact on the left atrial wall. In some examples, the points of contact between the docking device 300 and the left atrial wall can form a plane that is approximately parallel to a plane of the native mitral valve.

In some examples, the stabilization turn 310 can have an atrial portion 310a in connection with the proximal turn 308p of the central region 308, a stabilization portion 310c adjacent to the proximal end 302p of the coil 302, and an ascending portion 310b located between the atrial portion 310a and the stabilization portion 310c. In some examples, both the atrial portion 310a and the stabilization portion 310c can be generally parallel to the helical turns in the central region 308, whereas the ascending portion 310b can be oriented to be angular relative to the atrial portion 310a and the stabilization portion 310c. For example, the ascending portion 310b can be disposed an angle of about 45 degrees to about 90 degrees (inclusive) relative to one or both of the ascending portion 310b and the stabilization portion 310c. In some examples, the stabilization portion 310c can define a plane that is substantially parallel to a plane defined by the atrial portion 310a. A boundary 307 (shown in dashed line in FIG. 6A) between the ascending portion 310b and the stabilization portion 310c can be determined as a location where the ascending portion 310b intersects the plane defined by the stabilization portion 310c. The curvature of the stabilization turn 310 can be configured so that the atrial portion 310a and the stabilization portion 310c are disposed on approximately opposite sides when the docking device 300 is fully expanded. When implanting the docking device 300 at the native mitral valve location, the atrial portion 310a can be configured to abut the posterior wall of the left atrium and the stabilization portion 310c can be configured to flare out and press against the anterior wall of the left atrium (see e.g., FIGS. 12A and 13A).

As noted above, the leading turn 306 can have a larger radial dimension than the helical turns in the central region 308. In some examples, the leading turn 306 can help more easily guide the coil 302 around and/or through the chordae tendineae and/or adequately around all native leaflets of the native valve (e.g., the native mitral valve, tricuspid valve, etc.). For example, once the leading turn 306 is navigated around the desired native anatomy, the remaining coil (such as the functional turns) of the docking device 300 can also be guided around the same features. In some examples, the leading turn 306 can be a full turn (i.e., rotating about 360 degrees). In some examples, the leading turn 306 can be a partial turn (e.g., rotating between about 180 degrees and about 270 degrees). As described further below in reference to FIGS. 11A-13B, when a prosthetic valve is radially expanded within the central region 308 of the coil 302, the functional turns in the central region 308 can be further radially expanded. As a result, the leading turn 306 can be pulled in the proximal direction and become a part of the functional turn in the central region 308.

In some examples, the coil 302 can include the interior coil member 311 and a coil member cover 312 that is at least partially surrounds the coil member 311. As shown in FIGS. 7A and 7B, the coil member cover 312 can have a tubular shape and thus can also be referred to as a “tubular member.” In some examples, the tubular member 312 can cover an entire length of the coil member 311. In some examples, the tubular member 312 covers only selected portion(s) of the coil member 311.

In some examples, the tubular member 312 can be coated on and/or bonded on the coil member 311. In some examples, the tubular member 312 can be a cushioned, padded-type layer protecting the coil member 311. The tubular member 312 can be constructed of various native and/or synthetic materials. In one particular example, the tubular member 312 can include expanded polytetrafluoroethylene (ePTFE). In some examples, the tubular member 312 is configured to be fixedly attached to the coil member 311 (e.g., by means of textured surface resistance, suture, glue, thermal bonding, or any other means) so that relative axial movement between the tubular member 312 and the coil member 311 is restricted or limited.

In some examples, the docking device 300 can have one or more seating markers. For example, FIGS. 6A and 6B show a proximal seating marker 321p and a distal seating marker 321d, wherein the proximal seating marker 321p is positioned proximal relative to the distal seating marker 321d. Both the proximal and distal seating markers 321p, 321d can have predefined locations relative to the coil 302. As shown, both the proximal and distal seating markers 321p, 321d can be disposed distal to the ascending portion 310b, e.g., at the atrial portion 310a of the coil 302 and/or proximate to the proximal end 304p of the guard member 304.

In some examples, the proximal and distal seating markers 321p, 321d can include a radiopaque material so that the markers can be visible under fluoroscopy, such as during an implantation procedure of the docking device 300 and/or a prosthetic valve. As described further below, the seating markers 321p, 321d can be used to mark the proximal and distal boundaries of a segment of the coil 302 where the proximal end 304p of the guard member 304 can be positioned when deploying the docking device 300.

In some examples, the seating markers 321p, 321d can be disposed on the tubular member 312. In some examples, the seating markers 321p, 321d can be disposed on the coil member 311 and covered by the tubular member 312. In some examples, the seating markers 321p, 321d can be disposed on different layers relative to each other. For example, one of the seating markers (e.g., 321p) can be disposed outside the tubular member 312, whereas another seating marker (e.g., 321d) can be disposed directly on the coil member 311 and covered by the tubular member 312.

In some examples, a segment of the coil 302 located between the proximal seating marker 321p and the distal seating marker 321d can have an axial length in the range of about 2 mm to about 7 mm, or about 3 mm to about 5 mm. In one specific example, the axial length of the coil segment between the proximal seating marker 321p and the distal seating marker 321d is about 4 mm. Further, in some examples, an axial distance between the proximal seating marker 321p and a distal end of the ascending portion 310b can have an axial length in the range of about 10 mm to about 30 mm, or about 15 mm to about 25 mm. In one specific example, the axial distance between the proximal seating marker 321p and the distal end of the ascending portion 310b is about 20 mm.

Although two seating markers 321p, 321d are shown in FIGS. 6A and 6B, it is to be understood that the docking device 300 can include fewer or additions seating markers and/or the seating markers can be disposed at alternate or additional locations. In some examples, the docking device 300 can have only one seating marker (e.g., 321p). In some examples, one or more additional seating markers can be placed between the proximal and distal seating markers 321p, 321d. As noted above, the proximal end 304p of the guard member 304, 604 can be positioned between the proximal and distal seating markers 321p, 321d when deploying the docking device 300. As such, these additional seating markers can function as a scale to indicate a precise location of the proximal end 304p of the guard member 304, 604 on the coil 302. In some examples, the docking device 300 can include additional seating markers at other locations, such as including one or more seating markers positioned proximate to the distal end 304d of the guard member 304, 604 or other locations of the guard member 304, 604.

In some examples, as shown in FIGS. 6A and 6B, when the docking device 300 is in the deployed configuration, the guard member 304, 604 can be configured to cover an exterior surface of at least a portion of the stabilization turn 310 (e.g., at least a portion of the atrial portion 310a) and an exterior surface of at least at least a portion of the central region 308 of the coil 302, such as at least a portion of the proximal turn 308p.

In some examples, the guard member 304, 604 can further extend over an exterior surface at least a portion of the intermediate turn(s) 308m. In some examples, the guard member 304, 604 can extend over an exterior surface of the entirety of the coil 302. In some examples, the guard member can be discontinuous, and extend over exterior surfaces of discrete portions of the coil 302, such as over opposing sides of the proximal turn 308p (see, e.g., a discontinuous guard member 304c, 604c shown in FIGS. 13A and 13B and discussed further below). In some examples, the guard member 304, 604 can additionally be disposed on or extend over interior, proximal, and/or distal surfaces of the coil 302 or circumferentially around the coil member 311 and the coil member cover 312.

FIGS. 7A-7E illustrate an exemplary guard member 304. As shown in FIGS. 7A-7B, the guard member 304 can be transitioned between a radially expanded or released or extended configuration 321 where the dock sleeve 220 is removed (FIG. 7A) and a compressed configuration 323 where the guard member 304, the coil member cover 312, and the coil member 311 are disposed inside of the dock sleeve 220 (FIG. 7B). As discussed above, in some examples, the docking device 300 can be configured to be disposed inside of the dock sleeve 220 and have the guard member 304 in the compressed configuration during delivery of the docking device to the implant location. After positioning at the implant location, the dock sleeve 220 can be withdrawn or otherwise axially moved relative to the coil 302 to allow the guard member 304 to expand or decompress or radially extend.

As can be seen in FIGS. 7C-7E, in some examples, the guard member 304 can be comprised of a plurality of fibers, filaments, yarns, or strands (e.g., pile yarns) 320 inserted between and/or captured between two core members 322. In some examples, the guard member 304 can include additional axial core members (such as, for example, three or four or more axial core members). As can be seen in the schematic illustration of FIG. 8A, the core members 322 are twisted or intertwined with each other in order to form intersecting (cross-over) points 326 with receiving sections 328 disposed therebetween, which can have the fibers 320 inserted therethrough. The fibers 320 can be disposed in an approximately perpendicular orientation relative to the core members 322, such that the fibers 320 extend radially outwardly from the core members 322 and form a concentric pile surface 324. Thus, a width or diameter of the guard member 304 can be defined by a length/defined by or generally corresponding to a length of the fibers 320.

As best seen in FIG. 7A, because of the attachment of the guard member 304 to the exterior surface of the coil 302, the fibers 320 on an interior portion of the guard member 304 are at least partially compressed against the exterior surface of the coil 302, and, on an opposing exterior portion of the guard member 304, the fibers 320 extends radially outward from the core members 322 and the surface of the coil 302.

In some examples, the fibers 320 can be thinner than the core members 322 and can be comprised of one or more types of fibrous materials, such as polyethylene terephthalate (PET), nylon, or other biocompatible materials. The material of the fibers 320 can be flexible such that the fibers can be folded, pressed, or bunched when the dock sleeve 220 is extended over the guard member 304 (e.g., when the coil member 311, the coil member cover 312, and the guard member 304 are inserted through the dock sleeve 220) to transition the guard member 304 from the radially extended configuration 321 to the compressed configuration 323. Further, the material of the fibers 320 can be resilient so that when the dock sleeve 220 is removed, the fibers 320 extend radially outward from the core members 322 and return to a generally linear form in order to transition the guard member 304 from the compressed configuration 323 to the radially extended configuration 321.

In some examples, the core members 322 can be thicker than the fibers 320 and can be comprised a woven material, such as polyethylene terephthalate (PET), nylon, or other inorganic materials, or can be comprised of a metallic material, such as stainless steel, cobalt, nitinol, or other metal. In some examples, where the core members are comprised of a metallic material, the guard member can have a more rigid overall structure that can be flexed or bent into a specified shape. In some examples, where the core members are comprised of woven material, the guard member can have a more flexible overall structure and may not retain a specified shape without being attached to the coil.

It will be appreciated that the schematic depiction in FIG. 7C of the guard member 304 shows the core members 322 and the fibers 320 is a loosened state for illustrative purposes. However, in order to capture and retain the fibers 320, the core members 322 can be wound or intertwined tightly such that there is minimal or no space between the core members. In other words, the core members 322 can be wrapped tightly around the fibers 320. Further, in some examples, the core members 322 can have a higher twist density (i.e., a greater number of cross-over points 326) to improve retainment of the fibers 320. In some examples, the core members 322 can have a lower twist density (i.e., a lower number of cross-over points 326) that provides a reduced compression profile of the guard member 304 when in the compressed state 323.

FIGS. 7D and 7E show exemplary guard members 304a and 304b. As can be seen therein, a first exemplary guard member 304a includes two core members 322a and a plurality of fibers 320a retained therebetween (FIG. 7D), and a second exemplary guard member 304b includes two core members 322b and a plurality of fibers 320b retained therebetween (FIG. 7E). The fibers 320a have a relatively lower fiber density (i.e., a lower number of fibers) as compared to a higher fiber density the fibers 320b. Further, the fibers 320a have a relatively shorter length l₁ as compared to the fibers 320b, which have a longer length l₂.

It will be appreciated that increased fiber density and/or a longer length of the fibers may improve function of a guard member in limiting or preventing PVL. However, a lower fiber density and/or a shorter length of the fibers may improve (reduce) the compression profile of the guard member, thereby enabling easier implantation of the docking device. Accordingly, an optimized fiber density and length of the fibers can be selected to provide one or more of sufficient limiting of PVL and/or a sufficient compression profile of the guard member.

Although FIGS. 7C-7D show the plurality of fibers having a uniform length and density, in some examples, the fibers of a guard member can have varying lengths and/or the fiber density can be varied along the length of the guard member.

FIGS. 8A-8D illustrate an exemplary guard member 604. As shown in FIGS. 8A-8B, the guard member 604 can be transitioned between a radially expanded or released or extended configuration 621 where the dock sleeve 220 is removed (FIG. 8A) and a compressed configuration 323 where the guard member 604, the coil member cover 312, and the coil member 311 are disposed inside of the dock sleeve 220 (FIG. 8B). As discussed above, in some examples, the docking device 300 can be configured to be disposed inside of the dock sleeve 220 and have the guard member 604 in the compressed configuration during delivery of the docking device to the implant location. After positioning at the implant location, the dock sleeve 220 can be withdrawn or otherwise axially moved relative to the coil 302 to allow the guard member 604 to expand or decompress or radially extend.

As can be seen in FIGS. 8A-8B, in some examples, the guard member 604 can be comprised of a plurality of fibers, filaments, yarns, or strands (e.g., pile yarns) 620 that extend from one surface of a base layer 622. In some examples, the fibers 620 are attached to the base layer 622 (such as, for example, via an adhesive or being threaded through the base layer). In some examples, the fibers 620 are integral to the base layer 622 (such as, for example, being woven with the base layer, being extruded from the base layer, or being embedded in the base layer (e.g., embedded within a material during forming of the base layer)). In some examples, the base layer 622 is generally flat on its opposing surface and does not include fibers extending therefrom. In some examples, the fibers 620 can be disposed in an approximately perpendicular orientation relative to the base layer 622, such that the fibers 620 extend perpendicular to the base layer 622 and form a relatively flat pile surface 624 (when disposed on a flat surface). Thus, a width or thickness of the guard member 604 can be defined by a length/defined by or generally corresponding to a length of the fibers 620.

As can be seen in the example of FIG. 8A, when the guard member 604 is attached to the coil 302, the base layer 622 can be overlaid on the curved exterior surface of the coil 302, thereby giving the base layer 622 a curved configuration. Further, when the base layer 622 is in the curved configuration, the plurality of fibers 620 can extend radially relative to the coil member 311 and the coil member cover 312.

In some examples, the fibers 620 can be comprised of one or more types of thin fibrous materials, such as polyethylene terephthalate (PET), nylon, or other biocompatible materials. The material of the fibers 620 can be flexible such that the fibers can be folded, pressed, or bunched when the dock sleeve 220 is extended over the guard member 604 (such as, for example, when the coil member, the coil member cover 312, and the guard member 604 are inserted through the dock sleeve 220) to transition the guard member 604 from the radially extended configuration 621 to the compressed configuration 623. Further, the material of the fibers 620 can be resilient so that when the dock sleeve 220 is removed, the fibers 320 extend radially outward from the base layer 622 and return to a generally linear form in order to transition the guard member 604 from the compressed configuration 623 to the radially extended configuration 621.

In some examples, the base layer 622 can be thicker than the fibers 620 and can be comprised a woven material, such as woven polyethylene terephthalate (PET), nylon, or other inorganic material fibers, or can be comprised of a metallic material, such as stainless steel, cobalt, nitinol, or other metal, or can be comprised of a flexible sheet, such as, for example a polymeric material sheet comprising PTFE, PET, polypropylene, polyamide, polyetheretherketone (PEEK), or other polymeric materials or combinations thereof. In some examples, where the base layer is comprised of a metallic material, the guard member can have a more rigid overall structure that can be flexed or bent into a specified shape. In some examples, where the base layer is comprised of woven material and/or a flexible polymeric sheet, the guard member can have a more flexible overall structure and may not retain a specified shape without being attached to the coil.

FIGS. 8C and 8D show exemplary guard members 604a and 604b. As can be seen therein, a first exemplary guard member 604a includes a base layer 622a and a plurality of fibers 620a extending therefrom (FIG. 8C), and a second exemplary guard member 604b includes a base layer 622b and a plurality of fibers 620b extending therefrom (FIG. 8D). The fibers 620a have a relatively lower fiber density (i.e., a lower number of fibers) as compared to a higher fiber density the fibers 620b. Further, the fibers 620a have a relatively shorter length l₃ as compared to the fibers 620b, which have a longer length l₄.

It will be appreciated that increased fiber density and/or a longer length of the fibers may improve function of a guard member in limiting or preventing PVL. However, a lower fiber density and/or a shorter length of the fibers may improve (reduce) the compression profile of the guard member, thereby enabling easier implantation of the docking device. Accordingly, an optimized fiber density and length of the fibers can be selected to provide one or more of sufficient limiting of PVL and/or a sufficient compression profile of the guard member.

Although FIGS. 8C and 8D show the plurality of fibers having a uniform length and density, in some examples, the fibers of a guard member can have varying lengths and/or the fiber density can be varied along the length of the guard member.

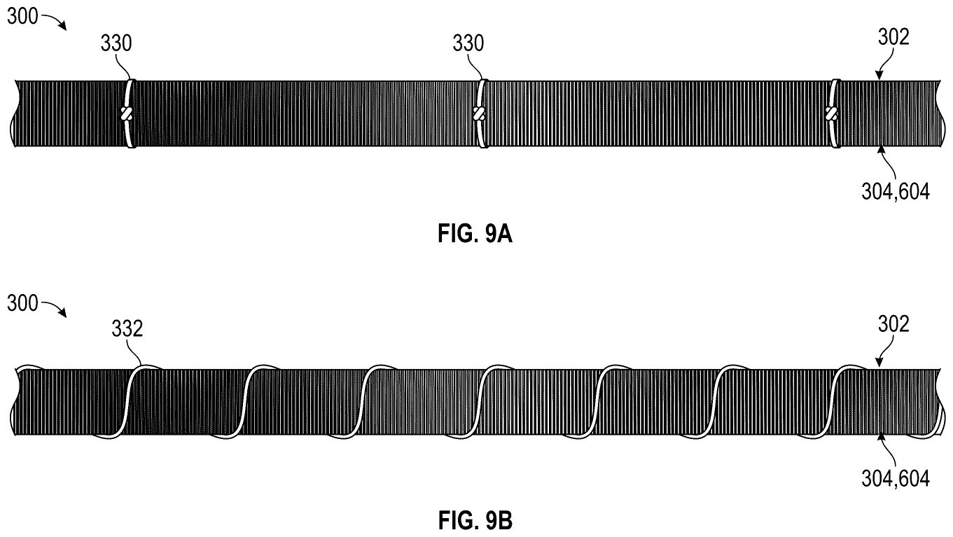

FIGS. 9A and 9B schematically illustrate exemplary attachment mechanisms for attachment of the guard member 304, 604 to the coil 302 of the docking device 300. In a first example shown in FIG. 9A, the guard member 304, 604 can be attached via discrete knotted sutures 330 disposed at specified increments along the length of the guard member 304, 604. In one specific example, a suture 330 encircles the coil member cover 312 (and the coil member 311 disposed therein) and the guard member 304, 604 and additional sutures 330 are disposed at 3 mm increments along the length of the guard member. In some examples, the sutures 330 can be disposed at shorter increments (e.g., 1 mm increments), thereby providing improved securing of the guard member 304, 604 to coil 302. In some examples, the sutures 330 can be disposed at greater increments (e.g., 5 mm increments), thereby reducing a compression profile of the guard member 304, 604.

In a second example shown in FIG. 9B, the guard member 304, 604 can be attached via a continuous wrapped suture 332 along the length of the guard member. In one specific example, the suture 332 encircles the coil member cover 312 (and the coil member 311 disposed therein) and the guard member 304, 604, and is wrapped therearound at 1 mm increments along the length of the guard member. It will be appreciated that although not specifically shown, the ends of the suture 332 can be knotted so that the suture does not unwind or unravel from the guard member 304, 604 and the coil 302. In some examples, the sutures 332 can be wrapped at shorter increments (e.g., 0.5 mm increments), thereby providing improved securing of the guard member to coil 302. In some examples, the sutures 330 can be wrapped at greater increments (e.g., 3 mm increments), thereby reducing a compression profile of the guard member 304, 604.

In some examples, a guard member 304, 604 can be attached via two or more wrapped sutures. For example, a guard member can be attached via three wrapped sutures that respectively extend over approximately one third of the length of the guard member. In some examples, a guard member can be attached via two wrapped sutures that extend of the entire length of the guard member and overlap with each other. In some examples, a guard member can be attached via a combination of discrete knotted sutures and wrapped sutures. In some examples, the guard member can be attached via a different attachment mechanism, such as via an adhesive. In some examples an adhesive can be used in combination with a suture attachment mechanism.

It will be appreciated that FIGS. 9A and 9B show the location of the sutures as disposed over or on top of the fibers of the guard member 304, 604 for illustrative purposes, such as demonstrating the suture pattern and configuration of the sutures. In practice, however, the sutures can be tightly wrapped around the guard member 304, 604 (for example, tightly wrapped to the core members 322 or the base layer 622) and the coil 302, and the fibers 320, 620 can extend around and/or cover the sutures, for example, when the guard member 304, 604 is in the expanded or radially extended configuration 321, 621.

In some examples, features of the guard member can be selected to balance profile density, retention of the fibers, and/or the ability of the guard to prevent or limit PVL. Specifically, in some examples, one or more of an average length of the fibers, a density of the fibers, an average thickness the fibers, a material of the fibers, a twist density of the at least two axial core members, a material of the at least two axial core members, a thickness of the at least two axial core members, and/or wrap or suture frequency can be selected to minimize a compression profile of the guard member in the compressed state, maximize retention of the fibers, and/or improve function in limiting or preventing PVL.

### Exemplary Prosthetic Valves

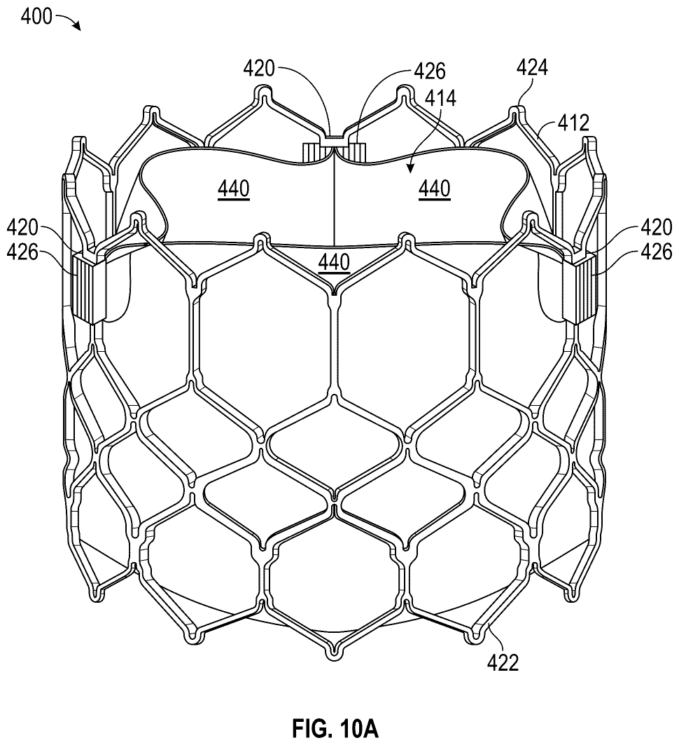

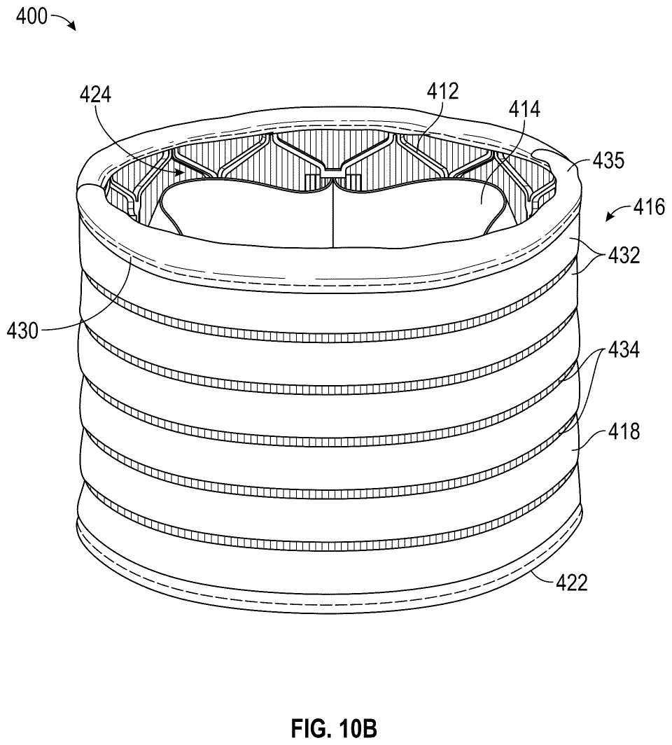

FIGS. 10A-11B show a prosthetic valve 400, according to one example. The prosthetic valve 400 (which may be used in a similar manner as the prosthetic valve 62 described above with respect to FIGS. 3A-4) can be adapted to be implanted, with or without a docking device, in a native valve annulus, such as the native mitral valve annulus, native aortic annulus, native pulmonary valve annulus, etc. The prosthetic valve 400 can include a stent or frame 412, a valvular structure 414, and a valve cover 416 (the valve cover 416 is removed in FIG. 10A to illustrate the frame structure).

The valvular structure 414 can include three leaflets 440, collectively forming a leaflet structure (although a greater or fewer number of leaflets can be used), which can be arranged to collapse in a tricuspid arrangement. The leaflets 440 are configured to permit the flow of blood from an inflow end 422 to an outflow end 424 of the prosthetic valve 400 and block the flow of blood from the outflow end 424 to the inflow end 422 of the prosthetic valve 400. The leaflets 440 can be secured to one another at their adjacent sides to form commissures 426 of the leaflet structure. The lower edge of valvular structure 414 desirably has an undulating, curved scalloped shape.

By forming the leaflets 440 with this scalloped geometry, stresses on the leaflets 440 can be reduced, which in turn can improve durability of the prosthetic valve 400. Moreover, by virtue of the scalloped shape, folds and ripples at the belly of each leaflet 440 (the central region of each leaflet), which can cause early calcification in those areas, can be eliminated or at least minimized. The scalloped geometry can also reduce the amount of tissue material used to form leaflet structure, thereby allowing a smaller, more even crimped profile at the inflow end of the prosthetic valve 400. The leaflets 440 can be formed of pericardial tissue (e.g., bovine pericardial tissue), biocompatible synthetic materials, or various other suitable natural or synthetic materials as known in the art and described in U.S. Pat. No. 6,730,118, which is incorporated by reference herein.

The frame 412 can be formed with a plurality of circumferentially spaced slots, or commissure windows 420 (three in the illustrated example) that are adapted to mount the commissures 426 of the valvular structure 414 to the frame. The frame 412 can be made of any of various suitable plastically expandable materials (e.g., stainless steel, etc.) or self-expanding materials (e.g., Nitinol) as known in the art. When constructed of a plastically expandable material, the frame 412 (and thus the prosthetic valve 400) can be crimped to a radially compressed state on a delivery apparatus and then expanded inside a patient by an inflatable balloon or equivalent expansion mechanism. When constructed of a self-expandable material, the frame 412 (and thus the prosthetic valve 400) can be crimped to a radially compressed state and restrained in the compressed state by insertion into a valve sheath or equivalent mechanism of a delivery apparatus. Once inside the body, the prosthetic valve 400 can be advanced from the delivery sheath, which allows the prosthetic valve 400 to expand to its functional size.

Suitable plastically expandable materials that can be used to form the frame 412 include, without limitation, stainless steel, a nickel-based alloy (e.g., a cobalt-chromium or a nickel-cobalt-chromium alloy), polymers, or combinations thereof. In some examples, frame 412 can be made of a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight. It has been found that the use of MP35N to form the frame 412 can provide superior structural results over stainless steel. In particular, when MP35N is used as the frame material, less material is needed to achieve the same or better performance in radial and crush force resistance, fatigue resistances, and corrosion resistance. Moreover, since less material is required, the crimped profile of the frame can be reduced, thereby providing a lower profile valve assembly for percutaneous delivery to the treatment location in the body.

As shown in FIG. 10B, the valve cover 416 can include an outer portion 418 which can cover an entire outer surface of the frame 412. In some examples, the valve cover 416 can also include an inner portion 428 (shown in FIG. 11A) which can, in some examples, cover an entire inner surface of the frame 412, or in some examples, cover only selected portions of the inner surface of the frame 412. The valve cover 416 can extend over the outflow end 424 apices of the prosthetic valve 400 and can be affixed to the frame 412 by a variety of means, such as via sutures 430.

In some examples, a reinforcement strip or apex covering 435 can be disposed over the outflow end 424 portion of the valve cover 416 to protect the outflow end portion and/or prevent wear or tearing. In examples including the reinforcement strip 435, the sutures 430 can extend through the inner and outer layers of the reinforcement strip 435 (i.e., through respective edges of the reinforcement strip 435 that extend over the interior and exterior surfaces of the outflow end of the prosthetic valve 400) to secure the reinforcement strip 435 to the valve 400. Similarly, as can be seen in FIG. 11A, a reinforcement strip or apex covering 437 can be disposed over the inflow end 422 portion of the valve cover 416 to protect the outflow end portion and/or prevent wear or tearing. Sutures 431 can extend through the inner and outer layers of the reinforcement strip 437 (i.e., through respective edges of the reinforcement strip 437 that extend over the interior and exterior surfaces of the inflow end 422 of the prosthetic valve 400) to secure the reinforcement strip 437 to the valve 400.

As described herein, the valve cover 416 can be configured to prevent or limit PVL between the prosthetic valve 400 and the native valve, to protect the native anatomy, to promote tissue ingrowth, among some other purposes. For mitral valve replacement, due to the general D-shape of the mitral valve and relatively large annulus compared to the aortic valve, the valve cover 416 can act as a seal around the prosthetic valve 400 (e.g., when the prosthetic valve 400 is sized to be smaller than the annulus) and allows for smooth coaptation of the native leaflets against the prosthetic valve 400.

In some examples, the valve cover 416 can include a material that can be crimped for transcatheter delivery of the prosthetic valve 400 and is expandable to prevent or limit PVL around the prosthetic valve 400. Examples of possible materials include foam, cloth, fabric, one or more synthetic polymers (e.g., polyethylene terephthalate (PET), polytetrafluoroethylene (PTFE), expanded polytetrafluoroethylene (ePTFE), etc.), organic tissues (e.g., bovine pericardium, porcine pericardium, equine pericardium, etc.), and/or an encapsulated material (e.g., an encapsulated hydrogel).

In some examples, the valve cover 416 can be made of a woven cloth or fabric possessing a plurality of floated yarn sections 432 (e.g., protruding or puffing sections, also referred to as “floats” hereinafter). Details of exemplary covered valves with a plurality of floats 432 are further described in U.S. Patent Publication Nos. US2019/0374337, US2019/0192296, and US2019/0046314, which are each incorporated by reference herein. In some examples, the float yarn sections 432 are separated by one or more horizontal bands 434. In some examples, the horizontal bands 434 can be constructed via a leno weave, which can improve the strength of the woven structure. In some examples of the woven cloth, vertical fibers (e.g., running along the longitudinal axis of the prosthetic valve 400) can include a yarn or other fiber possessing a high level of expansion, such as a texturized weft yarn, while horizontal fibers (e.g., running circumferentially around the prosthetic valve 400) in a leno weave can include a low expansion yarn or fiber. In some examples, the horizontal bands 434 can be sutures extending through the material of the floats 432.

In some examples, the valve cover 416 can include a woven cloth resembling a greige fabric when assembled and under tension (e.g., when stretched longitudinally on a compressed valve prior to delivery of a prosthetic valve 400). When the prosthetic valve 400 is deployed and expanded, tension on floats 432 is relaxed allowing expansion of the floats 432. In some examples, the valve cover 416 can be heat set to allow floats 432 to return to an enlarged, or puffed, space-filling form. In some examples, the number and sizes of floats 432 can be optimized to provide a level of expansion to prevent PVL across the mitral plane (e.g., to have a higher level of expansion thickness) and/or a lower crimp profile (e.g., for delivery of the prosthetic valve). Additionally, the horizontal bands 434 can be optimized to allow for attachment of the valve cover 416 to the frame 412 based on the specific size or position of struts or other structural elements on the prosthetic valve 400.

Further details of the prosthetic valve 400 and its components are described, for example, in U.S. Pat. Nos. 9,393,110 and 9,339,384, which are each incorporated by reference herein. Additional examples of the valve cover are described in PCT Patent Application Publication No. WO/2020/247907 and U.S. Patent Publication No. US2019/0192296, previously incorporated by reference herein.

As described above and illustrated in FIGS. 11A and 11B, in some examples, the prosthetic valve 400 can be radially expanded and securely anchored within the docking device 300.

In some examples, the coil 302 of the docking device 300 in the deployed configuration can be movable between a first radially expanded configuration prior to the prosthetic valve 400 being radially expanded within the coil 302 and a second radially expanded configuration after the prosthetic valve 400 is radially expanded within the coil 302. For example, in FIGS. 6A and 6B the coil 302 is in the first radially expanded configuration, and in the example depicted in FIGS. 11A and 11B, the coil 302 is in the second radially expanded configuration since the prosthetic valve 400 is radially expanded therein.

In some examples, at least a portion of the coil 302, such as the central region 308, can have a larger diameter in the second radially expanded configuration than in the first radially expanded configuration (for example, a diameter the central region 308 can be increased by radially expanding the prosthetic valve 400 within the central region 308 of the coil 302). As the central region 308 increases in diameter when the coil 302 moves from the first radially expanded configuration to the second radially expanded configuration, the functional turns in the central region 308 and the leading turn 306 can rotate circumferentially (for example, in clockwise or counter-clockwise direction when viewed from the stabilization turn 310). Circumferential rotation of the functional turns in the central region 308 and the leading turn 306, which can also be referred to as “clocking,” can slightly unwind the helical coil or turns in the central region 308. Generally, the unwinding can be less than a turn (less than 360 degrees), or less than a half turn (less than 180 degrees). For example, the unwinding can be about 30 degrees and may be up to 90 degrees in certain circumstances. As a result, a distance between the proximal end 302p and the distal end 302d of the coil 302 measured along the central longitudinal axis of the coil 302 can foreshorten.

Further, an overlapping region between the proximal end 304p and the distal end 304d of the guard member 304, 604 may be reduced (so that the overlapping region has a shorter length), or eliminated (so that there is no overlap). For example, as depicted in FIGS. 11A and 11B, the overlapping region can be reduced or eliminated such that the proximal end 304p and the distal end 304d are approximately aligned, and the guard member 304, 604 extends circumferentially over the docking device 300 approximately 360 degrees when the prosthetic valve 400 is disposed and expanded within the docking device 300 and the docking device 300 is in the second radially expanded configuration.

FIGS. 12A and 12B illustrate the docking device 300 and the prosthetic valve 400 in an exemplary implanted state within a mitral valve 500. Specifically, in the present example, the prosthetic valve 400 is radially expanded within the mitral valve 500 while seated in the central region 308 of the coil 302. The functional turns in the central region 308 of the docking device 300 are radially expanded by the prosthetic valve 400 (that is, the coil 302 of the docking device 300 is expanded from the first radially expanded configuration to the second radially expanded configuration, as described above). As shown in FIG. 12A, in some examples, when the docking device 300 and the prosthetic valve 400 are implanted, a portion (e.g., approximately a first half) of the guard member 304, 604 can be disposed on the inflow side of the annulus of the mitral valve 500. Further, as shown in FIG. 12B, in some examples, another portion (e.g., approximately a second half) of the guard member 304, 604 can be disposed on the outflow side of the annulus of the mitral valve 500 and extend between commissures 502 of the mitral valve 500.

The radial tension between the prosthetic valve 400 and the central region 308 of the docking device 300 can securely hold the prosthetic valve 400 in place relative to the anatomy of the mitral valve 500. In addition, the guard member 304 can act as a seal between the docking device 300 and the anatomy of the mitral valve 500 to prevent or reduce PVL around the native annulus.

In some examples, the implanted prosthetic valve 400 and the docking device 300 may be particularly susceptible to PVL at the location of the native commissures 502 of the mitral valve 500. Accordingly, in some examples, the docking device 300 can include a reduced form guard member, such as a guard member 304c, 604c having two sections that are discontinuous and are configured to target the PVL susceptible regions of the mitral valve. As shown in FIG. 13B, each section of the guard member 304c can be configured to be disposed proximate to or at one of the commissures 502 on the outflow side of the mitral valve 500 when the docking device 300 and the prosthetic valve 400 are implanted therein. Further, as shown in FIG. 12A, when the docking device 300 and the prosthetic valve 400 are implanted within the mitral valve 500, the guard member is not disposed on the inflow side of the mitral valve 500.

It will be appreciated that, in some examples, a reduced form guard member can have other configurations, such as including more than two sections (such as, for example, including three, four or more sections) configured to be disposed on the inflow side and/or the outflow side of a native valve. In some examples, a reduced form guard member can be configured to prevent or limit PVL at regions of the native valve or the prosthetic valve that are particularly susceptible to PVL, while providing a reduced overall bulk of the PVL guard (relative to a continuous guard member) which can enable a reduced compression profile of the docking device.

It will be appreciated that any of the systems, devices, apparatuses, etc. herein can be sterilized (e.g., with heat/thermal, pressure, steam, radiation, and/or chemicals, etc.) to ensure they are safe for use with patients, and any of the methods herein can include sterilization of the associated system, device, apparatus, etc. as one of the steps of the method. Examples heat/thermal sterilization include steam sterilization and autoclaving. Examples of radiation for use in sterilization include gamma radiation, ultra-violet radiation, and/or electron beam. Examples of chemicals for use in sterilization include ethylene oxide, hydrogen peroxide, peracetic acid, formaldehyde, and/or glutaraldehyde. Sterilization with hydrogen peroxide may be accomplished using hydrogen peroxide plasma, for example.

It will be further appreciated that the treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Additional Examples of the Disclosed Technology

In view of the above described examples of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.
Example 1. A docking device configured for securing a prosthetic valve, the docking device comprising: a coil member configured to be transitioned from a delivery configuration to a coiled configuration, wherein the coil member, when in the coiled configuration, comprises a plurality of helical turns; and a guard member extending, relative to a central longitudinal axis of the coil member in the coiled configuration, circumferentially over at least a portion of one or more of the helical turns, wherein the guard member comprises a plurality of fibers configured to be transitioned from a compressed state and a radially extended state.
Example 2. The docking device of any example disclosed herein, particularly example 1, wherein the guard member extends over an exterior surface of the at least portion of the one or more helical turns of the coil member.
Example 3. The docking device of any example disclosed herein, particularly examples 1 or 2, wherein the guard member is attached to the coil member via one or more sutures.
Example 4. The docking device of any example disclosed herein, particularly example 3, wherein the one or more sutures comprise a plurality of sutures that are knotted around the guard member and the coil member at increments.
Example 5. The docking device of any example disclosed herein, particularly example 3, wherein the one or more sutures comprises a continuous wrap suture that wraps around the guard member and the coil member at increments.
Example 6. The docking device of any example disclosed herein, particularly examples 1-5, wherein, when the guard member is in the expanded state, the plurality of fibers extend radially outwardly from the two axial core members on an exterior surface of the guard member.
Example 7. The docking device of any example disclosed herein, particularly examples 1-6, wherein, when the guard member is in the compressed state, the plurality of fibers are compressed against an exterior surface of the coil by a dock sleeve of a delivery apparatus.
Example 8. The docking device of any example disclosed herein, particularly examples claims 1-7, wherein the coil member in the coiled configuration is configured to transition from a first radially expanded state to a second radially expanded state via expansion of the prosthetic valve within a central space of the plurality of helical turns of the coil member, and wherein the coil member has a first diameter in the first radially expanded state and a second diameter in the second radially expanded state, the second diameter greater than the first diameter.
Example 9. The docking device of any example disclosed herein, particularly example 8, wherein, when the coil member is in the first radially expanded state, the guard member extends circumferentially more than 360 degrees around the coil member in the coiled configuration.
Example 10. The docking device of any example disclosed herein, particularly example 9, wherein, when the coil member is in the first radially expanded state, the guard member extends circumferentially in a range of 361-400 degrees around the coil member in the coiled configuration.
Example 11. The docking device of any example disclosed herein, particularly examples claims 8-10, wherein, when the coil member is in the second radially expanded state, the guard member extends circumferentially less than 361 degrees around the coil member in the coiled configuration.
Example 12. The docking device of any example disclosed herein, particularly example 11, wherein, when the coil member is in the second radially expanded state, the guard member extends circumferentially in a range of 180-360 degrees around the coil member in the coiled configuration.
Example 13. The docking device of any example disclosed herein, particularly examples 1-12, wherein the coil member in the coiled configuration comprises a central portion and the plurality of helical turns within the central portion comprise a proximal turn, at least one intermediate turn, and a distal turn, and wherein the guard member extends over at least a portion of the proximal turn.
Example 14. The docking device of any example disclosed herein, particularly example 13, further comprising one or more seating markers disposed at a proximal region of the proximal turn.
Example 15. The docking device of any example disclosed herein, particularly example 14, wherein the one or more seating markers comprise a proximal seating marker and a distal seating marker, and wherein a proximal end of the guard member is disposed between the proximal seating marker and the distal seating marker.
Example 16. The docking device of any example disclosed herein, particularly examples 1-15, wherein the guard member is discontinuous and comprises at least two sections disposed on opposing sides of the coil member in the coiled configuration.
Example 17. The docking device of any example disclosed herein, particularly examples 1-16, wherein the guard member is discontinuous and comprises at least two sections disposed on opposing sides of the coil member in the coiled configuration.
Example 18. The docking device of any example disclosed herein, particularly example 17, wherein one or more of an average length of fibers in the plurality of fibers, a density of the plurality of fibers, an average thickness of fibers in the plurality of fibers, a material of the plurality of fibers, a twist density of the at least two axial core members, a thickness of the at least two axial core members, or a material of the at least two axial core members is selected to minimize a compression profile of the guard member in the compressed state.
Example 19. The docking device of any example disclosed herein, particularly examples 17 or 18, wherein one or more of an average length of filaments in the plurality of fiber, a density of the plurality of fibers, an average thickness of fibers in the plurality of fibers, a material of the plurality of fibers, a twist density of the at least two axial core members, a thickness of the at least two axial core members, or a material of the at least two axial core members is selected to retain the plurality of fibers between the at least two core members.
Example 20. The docking device of any example disclosed herein, particularly examples 17-19, wherein one or more of an average length of filaments in the plurality of fiber, a density of the plurality of fibers, an average thickness of fibers in the plurality of fibers, a material of the plurality of fibers, a twist density of the at least two axial core members, a thickness of the at least two axial core members, or a material of the at least two axial core members is selected to limit paravalvular leakage when the docking device is implanted.
Example 21. The docking device of any example disclosed herein, particularly examples 1-16, wherein the guard member further comprises a base layer.
Example 22. The docking device of any example disclosed herein, particularly example 21, wherein the base layer comprises a woven base layer the plurality of fibers are integral to the woven base layer and extend from a surface thereof.
Example 23. The docking device of any example disclosed herein, particularly example 21, wherein the base layer comprises a woven base layer the plurality of fibers are attached to the woven base layer and extend from a surface thereof.
Example 24. The docking device of any example disclosed herein, particularly example 21, wherein the base layer comprises a flexible sheet and the plurality of fibers are integral thereto and extend from a surface thereof.
Example 25. The docking device of any example disclosed herein, particularly example 21, wherein the base layer comprises a flexible sheet and the plurality of fibers are attached thereto and extend from a surface thereof.
Example 26. The docking device of any example disclosed herein, particularly examples 21-25, wherein one or more of an average length of fibers in the plurality of fibers, a density of the plurality of fibers, an average thickness of fibers in the plurality of fibers, a material of the plurality of fibers, a thickness of the base layer, or a material of the base layer is selected to minimize a compression profile of the guard member in the compressed state.
Example 27. An assembly comprising: a prosthetic valve comprising an annular frame configured to be transitioned from a radially compressed configuration and a radially expanded configuration; and a valvular member disposed within the annular frame; and a docking device configured for anchoring the prosthetic valve, the docking device comprising: a coil member configured to be transitioned from a delivery configuration to a coiled configuration, wherein the coil member comprises a plurality of helical turns when the coil member is in the coiled configuration; and a guard member extending, relative to a central longitudinal axis of the coil member in the coiled configuration, circumferentially over at least a portion of one or more of the helical turns, wherein the guard member comprises a plurality of fibers configured to be transitioned between a compressed state and a radially extended state.
Example 28. The assembly of any example disclosed herein, particularly example 27, wherein, when the guard member is in the compressed state, the plurality of fibers are compressed against an exterior surface of the coil member by a dock sleeve of a delivery apparatus.
Example 29. The assembly of any example disclosed herein, particularly examples 27 or 28, wherein, when the guard member is in the radially extended state, the plurality of fibers extend radially outward from the exterior surface of the coil member.
Example 30. The assembly of any example disclosed herein, particularly examples 27-29, wherein the guard member is attached to the coil member via one or more sutures.
Example 31. The assembly of any example disclosed herein, particularly examples 27-30, wherein the coil member in the coiled configuration is configured to transition from a first radially expanded state to a second radially expanded state via expansion of the prosthetic valve within a central space of the plurality of helical turns of the coil member, and wherein the coil member has a first diameter in the first radially expanded state and a second diameter in the second radially expanded state, the second diameter greater than the first diameter.
Example 32. The assembly of any example disclosed herein, particularly example 31, wherein, when the coil member is in the first radially expanded state, the guard member extends more than 360 degrees around the coil member in the coiled configuration.
Example 33. The assembly of any example disclosed herein, particularly example 32, wherein, when the coil member is in the first radially expanded state, the guard member extends circumferentially in a range of 361-400 around the coil member in the coiled configuration.
Example 34. The assembly of any example disclosed herein, particularly examples 31-33, wherein, when the coil member is in the second radially expanded state, the guard member extends circumferentially less than 361 degrees around the coil member in the coiled configuration.
Example 35. The assembly of any example disclosed herein, particularly any of examples 31-34, wherein, when the coil member is in the second radially expanded state, the guard member extends circumferentially in a range of 180-360 degrees around the coil member in the coiled configuration.
Example 36. The assembly of any example disclosed herein, particularly examples 27-35, wherein the coil member in the coiled configuration comprises a central portion and the plurality of helical turns within the central portion comprise a proximal turn, at least one intermediate turn, and a distal turn, and wherein the guard member extends over at least a portion of the proximal turn.
Example 37. A delivery assembly comprising: a docking device configured for anchoring a prosthetic valve, the docking device comprising: a coil member configured to be transitioned from a delivery configuration to a coiled configuration, the coil member, when in the coiled configuration, comprising a plurality of helical turns; and a guard member extending circumferentially relative to a central longitudinal axis of the coil member in the coiled configuration over at least a portion of each of one or more of the helical turns, wherein the guard member comprises a plurality of fibers, and wherein the plurality of fibers are configured to be transitioned between a compressed state and radially extended state; and a docking device delivery apparatus configured for transcatheter delivery of the docking device to a native heart valve, the docking device delivery apparatus comprising: a delivery shaft comprising a lumen configured to receive the coil member therethrough when the coil member is in the delivery configuration; and a dock sleeve configured to circumferentially and axially extend over at least a portion of the coil member corresponding to a location of the guard member and compress the plurality of fibers into the compressed state, and wherein the dock sleeve is further configured to move axially relative to the coil member to release the plurality of fibers and allow the fibers to transition from the compressed state to the radially extended state.
Example 38. The delivery assembly of any example disclosed herein, particularly example 37, wherein the guard member extends over an exterior surface of the at least portion of each of the one or more helical turns of the coil member.
Example 39. The delivery assembly of any example disclosed herein, particularly examples 37 or 38, wherein the guard member is attached to the coil member via one or more sutures.
Example 40. The delivery assembly of any example disclosed herein, particularly examples 37-39, wherein the coil member in the coiled configuration comprises a central portion and the plurality of helical turns within the central portion comprise a proximal turn, at least one intermediate turn, and a distal turn.
Example 41. The delivery assembly of any example disclosed herein, particularly example 40, wherein the guard member extends over at least a portion of the proximal turn.
Example 42. The delivery assembly of any example disclosed herein, particularly examples 40 or 41, further comprising one or more seating markers disposed on a proximal region of the proximal turn.
Example 43. The delivery assembly of any example disclosed herein, particularly examples 37-42, further comprising the prosthetic valve, the prosthetic valve comprising: an annular frame configured to be transitioned from a radially compressed configuration and a radially expanded configuration; and a valvular member disposed within the annular frame.
Example 44. The delivery assembly of any example disclosed herein, particularly example 43, wherein the docking device is configured to receive the prosthetic valve within a central space of the plurality of helical turns of the coil member and is configured for radial expansion of the prosthetic valve therein.
Example 45. The delivery assembly of any example disclosed herein, particularly example 44, wherein the guard member is configured to extend circumferentially more than 360 degrees around the coil member in the coiled configuration prior to radial expansion of the prosthetic valve.
Example 46. The delivery assembly of any example disclosed herein, particularly example 44, wherein the guard member is configured to extend circumferentially 360 degrees or less around the coil member in the coiled configuration after radial expansion of the prosthetic valve.
Example 47. A method for implanting a prosthetic implant, the method comprising:
deploying a docking device at a native valve, wherein the docking device deployed at the native valve comprises a coil and a guard member extending circumferentially around at least a portion of the coil on an exterior surface thereof, the guard member comprising a plurality of fibers retained between at least two intertwined axial core members; and transitioning the plurality of fibers of the guard member from a compressed state to a radially extended state.
Example 48. The method of any example disclosed herein, particularly example 47, wherein the coil deployed at the native valve comprises a stabilization turn, one or more functional turns, and an ascending portion located between the stabilization turn and the one or more functional turns.
Example 49. The method of any example disclosed herein, particularly example 35, wherein deploying the docking device comprises wrapping leaflets of the native valve with the one or more functional turns of the coil and resting the stabilization turn of the coil against a native wall around the native valve.
Example 50. The method of any example disclosed herein, particularly examples 48 or 49, wherein the guard member extends over at least a portion of a proximal one of the one or more functional turns and is adjacent to an annulus of the native valve.
Example 51. The method of any example disclosed herein, particularly example 50, wherein the native valve is a mitral valve, and wherein at least a portion of the guard member is adjacent to the annulus on a ventricular side thereof.
Example 52. The method of any example disclosed herein, particularly examples 47-51, wherein transitioning the plurality of fibers of the guard member from the compressed state to the radially extended state comprises removing a dock sleeve from at least a region of the docking device corresponding to a location of the guard member on the coil.
Example 53. The method of any example disclosed herein, particularly examples 47-52, further comprising deploying the prosthetic valve within a central space of the coil of the docking device.
Example 54. The method of any example disclosed herein, particularly example 53, wherein the deploying the docking device further comprises determining a location of a proximal end of the guard member by monitoring a radiopaque marker located on opposing sides of the proximal end of the guard member.
Example 55. The method of any example disclosed herein, particularly examples 53 or 54, wherein the deploying the prosthetic valve within the central space of the coil of the docking device comprises positioning the prosthetic valve in a radially compressed state within the central space and radially expanding the prosthetic valve from the compressed state to a radially expanded state, wherein the prosthetic valve in the radially expanded state presses against an interior surface of the coil of the docking device.
Example 56. The method of any example disclosed herein, particularly example 55, wherein the radially expanding the prosthetic valve comprises radially inflating a balloon within the prosthetic valve.
Example 57. The method of any example disclosed herein, particularly example 55, wherein radially expanding the prosthetic valve comprises releasing the prosthetic valve from a valve sheath so as to allow self-expansion of the prosthetic valve.
Example 58. The method of any example disclosed herein, particularly examples 55-57, wherein the radially expanding the prosthetic valve results in transitioning of the coil of the docking device from a first radially expanded state to a second radially expanded state, the coil having a first diameter in the first radially expanded state and a second diameter in the second radially expanded state, the second diameter greater than the first diameter.
Example 59. The method of any example disclosed herein, particularly example 58, wherein the guard member extends, relative to a longitudinal axis of the coil, more than 360 degrees circumferentially around the coil when the coil is in the first radially expanded state.
Example 60. The method of any example disclosed herein, particularly examples 58 or 59, wherein the guard member extends, relative to a longitudinal axis of the coil, approximately 360 degrees circumferentially around the coil when the coil is in the second radially expanded state.
Example 61. The method of any example disclosed herein, particularly example 58, wherein the guard member is discontinuous and comprises a first section and a second section disposed on opposing sides of the coil, and wherein, when the coil is in the second radially expanded state, the first section is aligned with a first commissure of the native valve and the second section is aligned with a second commissure of the native valve.
Example 62. The method of any example disclosed herein, particularly examples 47-61, further comprising sterilizing one or more components of the prosthetic implant.
Example 63. The method of any example disclosed herein, particularly examples 47-62, wherein the method is a simulation procedure.

The features described herein with regard to any example can be combined with other features described in any one or more of the other examples, unless otherwise stated. For example, any one or more of the features of one guard member can be combined with any one or more features of another guard member. As another example, any one or more features of one docking device can be combined with any one or more features of another docking device.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A docking device configured for securing a prosthetic valve, the docking device comprising:
a coil member configured to be transitioned from a delivery configuration to a coiled configuration, wherein the coil member, when in the coiled configuration, comprises a plurality of helical turns; and
a guard member extending, relative to a central longitudinal axis of the coil member in the coiled configuration, circumferentially over at least a portion of one or more of the helical turns;
wherein the guard member comprises a plurality of fibers configured to be transitioned from a compressed state and a radially extended state.

2. The docking device of claim 1, wherein the guard member extends over an exterior surface of the at least portion of the one or more helical turns of the coil member.

3. The docking device of claim 1, wherein the guard member is attached to the coil member via a plurality of sutures that are knotted around the guard member and the coil member at increments.

4. The docking device of claim 1, wherein the guard member is attached to the coil member via a continuous wrap suture that wraps around the guard member and the coil member at increments.

5. The docking device of claim 1, wherein the coil member in the coiled configuration is configured to transition from a first radially expanded state to a second radially expanded state via expansion of the prosthetic valve within a central space of the plurality of helical turns of the coil member, and wherein at least a portion of the coil member has a first diameter in the first radially expanded state and has a second diameter in the second radially expanded state, the second diameter greater than the first diameter.

6. The docking device of claim 5, wherein, when the coil member is in the second radially expanded state, the guard member extends circumferentially in a range of 180-360 degrees around the coil member in the coiled configuration.

7. The docking device of claim 1, wherein the coil member in the coiled configuration comprises a central portion and the plurality of helical turns within the central portion comprise a proximal turn, at least one intermediate turn, and a distal turn, and wherein the guard member extends over at least a portion of the proximal turn.

8. The docking device of claim 7, further comprising one or more seating markers disposed at a proximal region of the proximal turn.

9. The docking device of claim 8, wherein the one or more seating markers comprise a proximal seating marker and a distal seating marker, and wherein a proximal end of the guard member is disposed between the proximal seating marker and the distal seating marker.

10. The docking device of claim 1, wherein the guard member is discontinuous and comprises at least two sections disposed on opposing sides of the coil member in the coiled configuration.

11. The docking device of claim 1, wherein the guard member further comprises at least two intertwined axial core members and the plurality of fibers are retained between the at least two intertwined axial core members.

12. The docking device of claim 11, wherein one or more of an average length of filaments in the plurality of fiber, a density of the plurality of fibers, an average thickness of fibers in the plurality of fibers, a material of the plurality of fibers, a twist density of the at least two axial core members, a thickness of the at least two axial core members, or a material of the at least two axial core members is selected to retain of the plurality of fibers between the at least two core members.

13. An assembly comprising:
a prosthetic valve comprising:
an annular frame configured to be transitioned from a radially compressed configuration and a radially expanded configuration; and
a valvular member disposed within the annular frame; and
a docking device configured for anchoring the prosthetic valve, the docking device comprising:
a coil member configured to be transitioned from a delivery configuration to a coiled configuration, wherein the coil member comprises a plurality of helical turns when the coil member is in the coiled configuration; and
a guard member extending, relative to a central longitudinal axis of the coil member in the coiled configuration, circumferentially over at least a portion of one or more of the helical turns, wherein the guard member comprises a plurality of fibers configured to be transitioned between a compressed state and a radially extended state.

14. The assembly of claim 13, wherein, when the guard member is in the compressed state, the plurality of fibers are compressed against an exterior surface of the coil member by a dock sleeve of a delivery apparatus.

15. The assembly of claim 13, wherein, when the guard member is in the radially extended state, the plurality of fibers extend radially outward from the exterior surface of the coil member.

16. The assembly of claim 13, wherein the guard member is attached to the coil member via one or more sutures.

17. The assembly of claim 13, wherein the coil member in the coiled configuration is configured to transition from a first radially expanded state to a second radially expanded state via expansion of the prosthetic valve within a central space of the plurality of helical turns of the coil member, and wherein the coil member has a first diameter in the first radially expanded state and a second diameter in the second radially expanded state, the second diameter greater than the first diameter.

18. The assembly of claim 17, wherein, when the coil member is in the first radially expanded state, the guard member extends more than 360 degrees around the coil member in the coiled configuration.

19. The assembly of claim 17, wherein, when the coil member is in the second radially expanded state, the guard member extends circumferentially less than 361 degrees around the coil member in the coiled configuration.

20. The assembly of claim 19, wherein the coil member in the coiled configuration comprises a central portion and the plurality of helical turns within the central portion comprise a proximal turn, at least one intermediate turn, and a distal turn, and wherein the guard member extends over at least a portion of the proximal turn.
